(19) United States
(12) Patent Application Publication
(10) Pub. No.: US 2022/0080043 A1
(43) Pub. Date: Mar. 17, 2022

(54) OIL/SURFACTANT MIXTURES FOR SELF-EMULSIFICATION

(71) Applicant: GLAXOSMITHKLINE BIOLOGICALS SA, Rixensart (BE)

(72) Inventors: Rushit LODAYA, Boston, MA (US); Mansoor AMIJI, Boston, MA (US); Derek O'HAGAN, Rockville, MD (US)

(73) Assignee: GLAXOSMITHKLINE BIOLOGICALS SA, Rixensart (BE)

(21) Appl. No.: 17/423,927

(22) PCT Filed: Jan. 29, 2020
(86) PCT No.: PCT/US2020/015565

§ 371 Date: Jul. 19, 2021

Related U.S. Application Data
(60) Provisional application No. 62/798,742, filed on Jan. 30, 2019.

(51) Int. Cl. A61K 39/39; A61K 47/22; A61K 47/06

(52) U.S. Cl. CPC A61K 39/39; A61K 2039/55566; A61K 47/06; A61K 47/22

(57) ABSTRACT

Methods of manufacturing squalene and alpha-tocopherol-containing oil-in-water emulsions having small oil droplet particle sizes. Such emulsions being of use as vaccine adjuvants.

### TECHNICAL FIELD

This invention relates to improved methods of manufacturing alpha-tocopherol-containing oil-in-water emulsions having small oil droplet particle sizes. Such emulsions may be of use as vaccine adjuvants. The invention also relates to emulsions which may be prepared by the improved methods and to compositions for use in the improved methods.

### BACKGROUND ART

The vaccine adjuvant known as ‘MF59’ (WO90/14837; Podda, 2003; Podda, 2001) is a submicron oil-in-water emulsion of squalene, polysorbate 80 (also known as Tween 80™), and sorbitan trioleate (also known as Span 85™). It may also include citrate ions e.g. 10 mM sodium citrate buffer. The composition of the emulsion by volume can be about 5% squalene, about 0.5% polysorbate 80 and about 0.5% sorbitan trioleate. The adjuvant and its production are described in more detail in Vaccine Design: The Subunit and Adjuvant Approach (chapter 10), Vaccine Adjuvants: Preparation Methods and Research Protocols (chapter 12) and New Generation Vaccines (chapter 19). As described in O'Hagan, 2007, MF59 is manufactured on a commercial scale by dispersing sorbitan trioleate in the squalene, dispersing polysorbate 80 in an aqueous phase (e.g. citrate buffer), then mixing these two phases to form a coarse emulsion which is then microfluidised. The emulsion is typically prepared at double-strength (4.3% v/v squalene, 0.5% v/v polysorbate 80 and 0.5% v/v sorbitan trioleate) and is diluted 1:1 (by volume) with an antigen containing composition to provide a final adjuvanted vaccine composition. An adult human dose of MF59 contains 9.75 mg squalene, 1.17 mg polysorbate 80 and 1.17 mg sorbitan trioleate (O'Hagan, 2013).

The emulsion adjuvant known as ‘AS03’ (Garcon, 2012) is prepared by mixing an oil mixture (consisting of squalene and alpha-tocopherol) with an aqueous phase (polysorbate 80 and buffer), followed by microfluidisation (WO2006/100109). AS03 is also typically prepared at double-strength with the expectation of dilution by an aqueous antigen containing composition. An adult human dose of AS03 contains 11.86 mg alpha-tocopherol, 10.69 mg squalene and 4.86 mg polysorbate 80 (Morel, 2011; Fox, 2009).

The emulsion adjuvant known as ‘AF03’ is prepared by cooling a pre-heated water-in-oil emulsion until it crosses its emulsion phase inversion temperature, at which point it thermoreversibly converts into an oil-in-water emulsion (US20070014805). The ‘AF03’ emulsion includes squalene, sorbitan oleate, polyoxyethylene cetostearyl ether and mannitol. The mannitol, cetostearyl ether and a phosphate buffer are mixed in one container to form an aqueous phase, while the sorbitan ester and squalene are mixed in another container to form an oily component. The aqueous phase is added to the oily component and the mixture is then heated to ˜60° C. and cooled to provide the final emulsion. The emulsion is initially prepared with a composition of 32.5% squalene, 4.8% sorbitan oleate, 6.2% polyoxyethylene cetostearyl ether and 6% mannitol, which is at least 4× final strength.

AS03 and MF59 adjuvants have been shown to augment the immune responses to 2 doses of an inactivated H7N9 influenza vaccine, with AS03-adjuvanted formulations inducing the highest titers (Jackson, 2015).

The presence of alpha-tocopherol in AS03 has been shown to enhance the magnitude of HBsAg antigen-specific adaptive responses in a mouse model (Morel, 2011).

As discussed above, traditional methods known in the art for producing emulsions suitable for use as adjuvants required either vigorous mechanical processes (such as homogenisation and microfluidisation) or relatively high temperatures (for example in a phase inversion temperature process) in order achieve the small oil droplet sizes required for adjuvant activity. The use of these processes is associated with several disadvantages e.g. high manufacturing costs.

WO2015/140138 and WO2016/135154 describe the preparation of oil/surfactant compositions, which when diluted with an aqueous phase spontaneously form oil-in-water emulsions having small droplet particle sizes, such emulsions can be used as immunological adjuvants. An adult human dose of SEA160 emulsion includes 7.62 mg squalene, 2.01 mg polysorbate 80 and 2.01 mg sorbitan trioleate. WO2015/140138 exemplifies the use of squalene and polysorbate 80 based compositions. Attempted replacement of squalene with sunflower oil or soybean oil and polysorbate 80 with polysorbate 20, sodium dodecyl sulfate or polyoxyethylene 10 lauryl ether lead to the conclusion that none of the alternative oils tested was a suitable substitute for squalene and none of the alternative surfactant components tested was useful.

Droplet size for self-emulsifying oil-in-water emulsion adjuvants has been shown to correlate with immune responses (Shah, 2014; Shah, 2015), with droplets of 160 nm diameter generating stronger immune responses than those of 20 nm or 90 nm.

Julianto, 2000, describes a self emulsifying vitamin E preparation comprising palm oil.

Mineral oil based emulsions comprising alpha-tocopherol have been investigated in a veterinary vaccine context (Franchini, 1991; Franchini, 1994).

There remains the need for new self-emulsifying oil/surfactant compositions which allow for the safe, convenient and cost-effective production of adjuvants on a commercially viable scale which display good immunological performance compared with adjuvants arising from conventional manufacturing approaches.

Accordingly, it is an object of the present invention to provide further and improved (e.g. simpler) methods for the production of submicron oil-in-water emulsions with novel compositions and improved immunological activities. In particular, it is an object of the present invention to provide methods that are suitable for use on a commercial scale and which do not require the use of processes involving vigorous mechanical treatment or significantly elevated temperatures.

### SUMMARY OF THE INVENTION

The inventors have surprisingly discovered that certain alpha-tocopherol-containing oil-in-water emulsions with small droplet sizes and low polydispersity index values (Pdl) can be formed without requiring either microfluidisation or heating to cause phase inversion, but rather by simple mixing of a suitable pre-mixed composition of oils and surfactant with aqueous material. The squalene/tocopherol/surfactant compositions of the invention can be mixed with an excess volume of aqueous material to spontaneously form oil-in-water emulsions with submicron oil droplets (and even with droplets having a diameter of 200 nm or less and Pdl of 0.3 or less, suitable for sterile filtration) which show good adjuvant activity, in some situations better than spontaneously forming emulsions without alpha-tocopherol, and especially comparable to the known alpha-tocopherol-containing emulsion AS03.

The present invention provides a composition comprising squalene, a tocopherol and a biocompatible metabolisable surfactant, wherein the squalene is 40% v/v or more of the composition, the tocopherol is 25% v/v or less of the composition, the surfactant is 60% v/v or less of the composition, which when mixed with an excess volume of substantially surfactant-free aqueous material, forms an adjuvant having an average oil particle diameter of 200 nm or less.

Also provided is a composition comprising squalene, a tocopherol and a biocompatible metabolisable surfactant, wherein the squalene is 50 to 70% v/v of the composition, the tocopherol is 10 to 20% v/v of the composition and the surfactant is 10 to 40% v/v of the composition.

Additionally provided is a method for preparing an oil-in-water emulsion adjuvant comprising squalene, a tocopherol, a biocompatible metabolisable surfactant and an aqueous component, said method comprising mixing a squalene, tocopherol and surfactant composition according to the invention with an excess volume of an aqueous component.

Further provided is an oil-in-water emulsion adjuvant composition comprising squalene, a tocopherol, a biocompatible metabolisable surfactant and an aqueous component, wherein the squalene is 40% v/v or more of the total amount of squalene, tocopherol and surfactant, the tocopherol is 25% v/v or less of the of the total amount of squalene, tocopherol and surfactant, the surfactant is 60% v/v or less of the total amount of squalene, tocopherol and surfactant and wherein the adjuvant has an average oil particle diameter of 200 nm or less.

Also provided is an oil-in-water emulsion adjuvant composition comprising squalene, a tocopherol, a biocompatible metabolisable surfactant and an aqueous component, wherein the squalene is 50 to 70% v/v of the total amount of squalene, tocopherol and surfactant, the tocopherol is 10 to 20% v/v of the total amount of squalene, tocopherol and surfactant and the surfactant is 10 to 40% v/v of the total amount of squalene, tocopherol and surfactant.

Vaccine compositions comprising an oil-in-water emulsion adjuvant of the present invention and an antigen or antigenic component, and kits of parts for the preparation of such vaccine compositions, are also provided by the present invention.

The present invention also provides a dried material (e.g. a lyophilisate) which, when reconstituted with an aqueous component, provides an oil-in-water emulsion according to the invention or vaccine comprising an oil-in-water emulsion according to the invention and an antigen or antigenic component.

### BRIEF DESCRIPTION OF THE FIGURES

FIG. 1—Ternary plot of squalene, tocopherol, polysorbate 80 emulsions prepared in Example 1 showing contours for the resulting particle diameter

FIG. 2—Ternary plot of squalene, tocopherol, polysorbate 80 emulsions prepared in Example 1 showing contours for the resulting particle polydispersity index

FIG. 3—HAI titers three weeks following the first immunisation with quadrivalent influenza vaccine as described in Example 3

FIG. 4—HAI titers three weeks following the second immunisation with quadrivalent influenza vaccine as described in Example 3

FIG. 5—IgG1 sub-type titers following immunisation with quadrivalent influenza vaccine as described in Example 3

FIG. 6—IgG2a sub-type titers following immunisation with quadrivalent influenza vaccine as described in Example 3

FIG. 7—IgG2b sub-type titers following immunisation with quadrivalent influenza vaccine as described in Example 3

FIG. 8—Frequency of CD4+ responses following immunisation with quadrivalent influenza vaccine as described in Example 3

FIG. 9—CD4+ T cell response shown as average of frequencies from 5 animals and classified as Th0, Th1, Th2 and Th17 type CD4 T cells following immunisation with quadrivalent influenza vaccine as described in Example 3

FIG. 10—Size distribution of formulation 36 and 22 before and after emulsion filtration through 0.22 um polyethersulfone filter as described in Example 4

FIG. 11—Size distribution of formulation 44 before and after emulsion filtration through 0.22 um polyethersulfone filter as described in Example 4

FIG. 12—pH and osmolality of formulation 36, 22 and 44 emulsions stored at 4° C., 25° C. or 50° C. for 10 weeks as described in Example 5

FIG. 13—Particle diameter and polydispersity index for formulation 36, 22 and 44 emulsions stored at 4° C., 25° C. or 50° C. for 10 weeks as described in Example 5

FIG. 14—Neutralising antibody titers three weeks following the first immunisation with CMV vaccine as described in Example 6

FIG. 15—Neutralising antibody titers three weeks following the second immunisation with CMV vaccine as described in Example 6

FIG. 16—Neutralising antibody titers three weeks following the third immunisation with CMV vaccine as described in Example 6

FIG. 17—Size distribution of formulation 44b as described in Example 7

FIG. 18—Protein integrity following lyophilisation as described in Example 7

FIG. 19—Neutralising antibody titers following immunisation with CMV vaccine as described in Example 8. Each bar represents geometric mean titers (GMT) with 95% confidence interval (CI). Significant differences are marked on the graph. Comparison with CMV alone is shown at the bottom, with AS03 in the middle and with lyophilized single vial at the top; where, ns=not significant, *=p<0.05, **=p<0.005, ***=p<0.0005 and ****=p<0.00005.

FIG. 20—Anti-CMV Penta IgG antibody titers in serum obtained three weeks post 2nd and 3rd immunization with CMV vaccine as described in Example 8. Each bar represents geometric mean titers (GMT) with 95% confidence interval (CI). Significant differences are marked on the graph. Comparison with CMV alone is shown at the bottom, with AS03 in the middle and with lyophilized single vial at the top; where, ns=not significant, *=p<0.05, **=p<0.005, ***=p<0.0005 and ****=p<0.00005.

FIG. 21—Antigen specific CD4+ T cells using ICS assay four weeks post following 3rd immunisation with CMV vaccine as described in Example 8. Each bar represents geometric mean titers (GMT) with 95% confidence interval (CI).

### DETAILED DESCRIPTION OF THE INVENTION

As mentioned above, the inventors have surprisingly discovered that alpha-tocopherol-containing oil-in-water emulsions with small droplet sizes and low polydispersity index values (Pdl) can be formed by simple mixing of a suitable pre-mixed composition of oils and surfactant with aqueous material. The pre-mixed compositions of the invention can be mixed with an excess volume of aqueous material to spontaneously form oil-in-water emulsions with submicron oil droplets (and even with droplets having a diameter of 200 nm or less and Pdl of 0.3 or less, suitable for sterile filtration) which show good adjuvant activity.

The present invention provides a composition comprising squalene, a tocopherol and a biocompatible metabolisable surfactant, wherein the squalene is 40% v/v or more of the composition, the tocopherol is 25% v/v or less of the composition, the surfactant is 60% v/v or less of the composition, which when mixed with an excess volume of substantially surfactant-free aqueous material, forms an adjuvant having an average oil particle diameter of 200 nm or less.

Also provided is a composition comprising squalene, a tocopherol and a biocompatible metabolisable surfactant, wherein the squalene is 50 to 70% v/v of the composition, the tocopherol is 10 to 20% v/v of the composition and the surfactant is 10 to 40% v/v of the composition.

Additionally provided is a method for preparing an oil-in-water emulsion adjuvant comprising squalene, a tocopherol, a biocompatible metabolisable surfactant and an aqueous component, said method comprising mixing a squalene, tocopherol and surfactant composition according to the invention with an excess volume of an aqueous component.

Further provided is an oil-in-water emulsion adjuvant composition comprising squalene, a tocopherol, a biocompatible metabolisable surfactant and an aqueous component, wherein the squalene is 40% v/v or more of the total amount of squalene, tocopherol and surfactant, the tocopherol is 25% v/v or less of the of the total amount of squalene, tocopherol and surfactant, the surfactant is 60% v/v or less of the total amount of squalene, tocopherol and surfactant and wherein the adjuvant has an average oil particle diameter of 200 nm or less.

Also provided is an oil-in-water emulsion adjuvant composition comprising squalene, a tocopherol, a biocompatible metabolisable surfactant and an aqueous component, wherein the squalene is 50 to 70% v/v of the total amount of squalene, tocopherol and surfactant, the tocopherol is 10 to 20% v/v of the total amount of squalene, tocopherol and surfactant and the surfactant is 10 to 40% v/v of the total amount of squalene, tocopherol and surfactant.

Vaccine compositions comprising an oil-in-water emulsion adjuvant of the present invention and an antigen or antigenic component, and kits of parts for the preparation of such vaccine compositions, are also provided by the present invention.

The present invention also provides a dried material (e.g. a lyophilisate) which, when reconstituted with an aqueous component, provides an oil-in-water emulsion according to the invention or vaccine comprising an oil-in-water emulsion according to the invention and an antigen or antigenic component.

Lodaya, 2019 describes some of the experimental data provided in the examples of the present application.

### Squalene/Tocopherol/Surfactant Compositions

According to the invention, processes for preparing oil-in-water emulsions make use of a squalene/tocopherol/surfactant composition. This composition is a mixture of squalene, tocopherol and a surfactant component, examples of which are discussed in more detail below.

The squalene, tocopherol and surfactant(s) in these components are ideally miscible in each other in the composition. The composition may be a squalene/tocopherol/surfactant dispersion, and if the squalene, tocopherol and surfactant phases are fully miscible in each other the composition will be in the form of a squalene/tocopherol/surfactant solution.

As emulsions of the invention are intended for pharmaceutical use, the surfactant(s) in the composition will typically be metabolisable (biodegradable) and biocompatible. The compositions and all components therein will typically be suitable for use as a pharmaceutical.

The composition ideally consists essentially of a squalene component, a tocopherol component and a surfactant component. In some embodiments, however, the composition can include component(s) in addition to the squalene, tocopherol and surfactant components. When further components are included, they should typically form less than 15% of the composition (by weight), more suitably less than 10%. For instance, in some embodiments the composition can include one or more excipients or pharmacologically active agent(s).

Squalene/tocopherol/surfactant compositions of the invention should be substantially free of aqueous components, and they may be anhydrous. A low water content is typically beneficial for stability. Suitably the squalene/tocopherol/surfactant compositions, whether formulated directly or prepared by drying of an emulsion, will contain 1% v/v water or less, such as 0.1% v/v or less, in particular 0.01% v/v or less and especially 0.001% v/v or less.

The proportions of the squalene component, tocopherol component and the surfactant component can vary. The squalene component will be 40% v/v or more of the composition, such as 50% or more and in particular 55% or more. The squalene component is desirably 90% v/v or less of the composition, such as 80% or less, in particular 70% or less and especially 65% or less. Suitably the squalene component is 50 to 70% v/v of the composition, such as 55 to 65%, in particular 57 to 63%, especially about 60% (such as 60%).

In order to ensure spontaneous formation of small droplet sizes a low tocopherol content is generally required. The tocopherol component will be 25% v/v or less of the composition, such as 20% or less. The tocopherol component is desirably 5% v/v or more of the composition, such as 10% or more. Suitably the tocopherol component is 5 to 25% v/v of the composition, such as 10 to 20%, in particular 12 to 18%, especially about 15% (such as 15%).

The surfactant component will be 60% v/v or less of the composition, such as 50% or less, in particular 40% or less, especially 30% or less. The surfactant component may be 10% v/v or more of the composition, such as 20% or more. Suitably the surfactant component is 15 to 35% v/v of the composition, such as 20 to 30%, in particular 22 to 28%, especially about 25% (such as 25%).

A desirable squalene/tocopherol/surfactant composition comprises squalene, alpha-tocopherol and surfactant, such as comprises squalene, alpha-tocopherol and polysorbate 80. More particularly desirable squalene/tocopherol/surfactant compositions consist essentially of squalene, alpha-tocopherol and surfactant, such consist essentially of as squalene, alpha-tocopherol and polysorbate 80.

### Squalene

Most fish contain metabolisable oils which may be readily recovered. A number of branched chain oils are synthesized biochemically in 5-carbon isoprene units and are generally referred to as terpenoids. Squalene, is a branched, unsaturated terpenoid ([(CH₃)₂C[═CHCH₂CH₂C(CH₃)]₂═CHCH₂₋]₂; C₃₀H₅₀; 2,6,10,15,19,23-hexamethyl-2,6,10,14,18,22-tetracosahexaene; CAS Registry Number 7683-64-9). Squalene is readily available from commercial sources or may be obtained by methods known in the art.

### Tocopherol

Any of the α, β, γ, δ, ε or ξ tocopherols can be used in the present invention, but α-tocopherols (also referred to herein as alpha-tocopherol) is typically used. D-α-tocopherol and DL-α-tocopherol can both be used. A desirable α-tocopherol is DL-α-tocopherol. Tocopherols are readily available from commercial sources or may be obtained by methods known in the art.

### The Surfactant Component(s)

The composition includes a surfactant component which is formed from one or more surfactant(s). Usually it will consist of one surfactant. In some embodiments the surfactant component will consist of more than one surfactant, such as a mixture consisting essentially of (such as consisting of) three surfactants, especially a mixture consisting essentially of (such as consisting of) two surfactants.

The surfactant component can include various surfactants, including ionic (cationic, anionic or zwitterionic) and/or non-ionic surfactants. The use of only non-ionic surfactants is often desirable, for example due to their pH independence. The invention can thus use surfactants including, but not limited to: the polyoxyethylene sorbitan ester surfactants (commonly referred to as the Tweens or polysorbates), such as polysorbate 20 and polysorbate 80, especially polysorbate 80; copolymers of ethylene oxide (EO), propylene oxide (PO), and/or butylene oxide (BO), sold under the DOWFAX™, Pluronic™ or Synperonic™ tradenames, such as linear EO/PO block copolymers, for example poloxamer 407 and poloxamer 188; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as polyoxyl 4 lauryl ether (Brij 30); polyoxyethylene-9-lauryl ether; sorbitan esters (commonly known as the Spans), such as sorbitan trioleate (Span 85), sorbitan monooleate (Span 80) and sorbitan monolaurate (Span 20); polyoxyethylene lauryl ether (Emulgen 104P) or a tocopherol derivative surfactant, such as alpha-tocopherol-polyethylene glycol succinate (TPGS). Many examples of pharmaceutically acceptable surfactants are known in the art for use in the composition and thus in the final emulsion e.g. see ‘Handbook of Pharmaceutical Excipients’ (eds. Rowe, Sheskey, & Quinn; 6th edition, 2009).

The surfactant(s) in the composition's surfactant component are suitably biocompatible and biodegradable. Thus the surfactant component will not, under normal usage, harm a mammalian recipient when administered, and can be metabolised so that it does not persist.

Surfactants of particular interest include polysorbates (e.g. polysorbate 20 or 80), sorbitan esters (e.g. sorbitan trioleate, sorbitan monooleate and sorbitan monolaurate), poloxamers (e.g. poloxamer 407 and poloxamer 188) and alpha-tocopherol PEG sugar esters (e.g. TPGS), which may be used individually, in combination with each other or in combination with other surfactants.

In some instances, a polysorbate, such as polysorbate 80, may be utilised in conjunction with a second surfactant, such as a poloxamer (e.g. poloxamer 407 and poloxamer 188) or an alpha-tocopherol PEG sugar ester (e.g. TPGS).

In some instances, polysorbate 80 is utilised individually as the surfactant component.

Surfactants can be classified by their ‘HLB’ (Griffin's hydrophile/lipophile balance), where a HLB in the range 1-10 generally means that the surfactant is more soluble in oil than in water, whereas a HLB in the range 10-20 means that the surfactant is more soluble in water than in oil. HLB values are readily available for surfactants of interest e.g. polysorbate 80 has a HLB of 15.0 and TPGS has a HLB of 13-13.2. Sorbitan trioleate has a HLB of 1.8.

When two or more surfactants are blended, the resulting HLB of the blend is typically calculated by the weighted average e.g. a 70/30 wt % mixture of polysorbate 80 and TPGS has a HLB of (15.0×0.70)+(13×0.30) i.e. 14.4. A 70/30 wt % mixture of polysorbate 80 and sorbitan trioleate has a HLB of (15.0×0.70)+(1.8×0.30) i.e. 11.04.

In general, the surfactant component has a HLB between 10 and 18, such as between 12 and 17, in particular 13 to 16. This can be typically achieved using a single surfactant or, in some embodiments, using a mixture of surfactants (e.g. of two surfactants, such as polysorbate 80 and second surfactant, such as TPGS).

Where the surfactant component includes more than one surfactant then at least one of them will typically have a HLB of at least 10 (e.g. in the range 12 to 17, or 13 to 16) and the other may have an HLB above 10 or a HLB below 10 (e.g. in the range of 1 to 9, or 1 to 4). In some embodiments the surfactant component comprises a first surfactant having an HLB value of from 1 to 5 and a second surfactant having an HLB value of from 13 to 17.

### The Aqueous Component

According to the invention, processes for preparing emulsions make use of an aqueous component, which is mixed with a squalene/tocopherol/surfactant composition of the invention. This aqueous component can be plain water (e.g. water for injection) or can include further components e.g. solutes. For instance, the aqueous component may include salts, which can be used to influence tonicity and/or to control pH. For instance, the salts can form a pH buffer e.g. citrate or phosphate salts, such as sodium salts. Typical buffers include: a phosphate buffer; a Tris buffer; a borate buffer; a succinate buffer; a histidine buffer; or a citrate buffer. Where a buffered aqueous component is used the buffer will typically be included in the 1-20 mM range.

The aqueous component can include solutes (which may be ionic or non-ionic) for influencing tonicity and/or osmolality. The tonicity can be selected to be approximately isotonic with human tissues. To control tonicity, the emulsion may comprise a physiological salt, such as a sodium salt. Sodium chloride (NaCl), for example, may be used at about 0.9% (w/v) (physiological saline). Other salts that may be present include potassium chloride, potassium dihydrogen phosphate, disodium phosphate, magnesium chloride, calcium chloride, etc. Non-ionic tonicifying agents can also be used to control tonicity. Monosaccharides classified as aldoses such as glucose, mannose, arabinose, and ribose, as well as those classified as ketoses such as fructose, sorbose, and xylulose can be used as non-ionic tonicifying agents in the present invention. Disaccharides such a sucrose, maltose, trehalose, and lactose can also be used. In addition, alditols (acyclic polyhydroxy alcohols, also referred to as sugar alcohols) such as glycerol, mannitol, xylitol, and sorbitol are non-ionic tonicifying agents useful in the present invention. Non-ionic tonicity modifying agents can be present at a concentration of from about 0.1% to about 10% w/v or about 1% to about 10% w/v, of the aqueous component depending upon the agent that is used.

Compositions for administration will usually have an osmolality in the range of 250 to 750 mOsm/kg, for example, the osmolality may be in the range of 250 to 550 mOsm/kg, such as in the range of 280 to 500 mOsm/kg. In a particular embodiment the osmolality may be in the range of 280 to 310 mOsm/kg. Osmolality may be measured according to techniques known in the art, such as by the use of a commercially available osmometer, for example the Advanced™ Model 2020 available from Advanced Instruments Inc. (USA).

Emulsions not directly intended for administration e.g. they are intended to first be mixed with a further liquid or dried composition containing an antigen or antigenic component, may themselves be hypo or hypertonic depending on the presence of components influencing tonicity and/or osmolality in said liquid or dried composition.

The aqueous component may comprise Pickering agents such as mannitol to reduce superficial tension.

The aqueous component ideally has a pH between 6 and 9 e.g. between 6.5 and 8.5, or between 6.0 and 7.5 or between 7.0 and 8.5. This pH range maintains compatibility with normal physiological conditions and, in certain instances, may be required in order to ensure stability of certain components of the emulsion.

Preferably, the aqueous component is substantially free from oil(s). Thus, on mixing with the squalene/tocopherol/surfactant composition to form an emulsion, substantially all of the oil in the emulsion should be sourced from the squalene/tocopherol/surfactant composition (e.g. at least 95% v/v, suitably at least 98% such as at least 99%). Preferably, the aqueous component is also substantially free from surfactant(s). Thus, on mixing with the squalene/tocopherol/surfactant composition to form an emulsion, substantially all of the surfactant in the emulsion should be sourced from the squalene/tocopherol/surfactant composition (e.g. at least 95% w/w, suitably at least 98% such as at least 99%). Most preferably, the aqueous component is substantially free from both oil(s) and surfactant(s).

In some embodiments the aqueous phase may comprise an antigen or antigenic component.

### Mixing

Unlike MF59 and AS03, emulsions of the invention can be prepared without requiring the use of homogenisers or microfluidisers. Unlike AF03, emulsions of the invention can be prepared without requiring heating up to >50° C. Instead, mixing the oil/surfactant composition with the aqueous phase can lead to spontaneous formation of a submicron emulsion even with only gentle agitation/mixing (e.g. by hand, such as by simple manual inversion).

Thus the invention provides a method for preparing an oil-in-water emulsion adjuvant having an average oil particle diameter of 200 nm or less and comprising squalene, a tocopherol, a biocompatible metabolisable surfactant and an aqueous component, said method comprising:
(i) providing a squalene/tocopherol/surfactant composition according to the invention;
(ii) providing an aqueous component;
(iii) combining the composition with an excess volume of the aqueous component to form a diluted composition; and
(iv) mixing the diluted composition to form an oil-in-water emulsion having an average oil particle diameter of 200 nm or less.

Also provided is a method for preparing an oil-in-water emulsion adjuvant having an average oil particle diameter of 200 nm or less and comprising squalene, a tocopherol, a biocompatible metabolisable surfactant and an aqueous component, said method comprising:
(iii) combining a squalene/tocopherol/surfactant composition according to the invention with an excess volume of an aqueous component to form a diluted composition; and
(iv) mixing the diluted composition to form an oil-in-water emulsion having an average oil particle diameter of 200 nm or less.

Step (iii) can take place by simple mixing of the squalene/tocopherol/surfactant composition with the aqueous component. Preferably it is achieved by adding the squalene/tocopherol/surfactant composition into the aqueous component. Step (iii) may sometimes comprise two separate steps: (a) initial mixing volumes of squalene/tocopherol/surfactant composition and aqueous component; and (b) diluting the mixture of squalene/tocopherol/surfactant composition and aqueous component with a further volume of an aqueous component to form the diluted composition. The steps (a) and (b) are preferably each achieved by adding the squalene/tocopherol/surfactant-containing material into an aqueous component.

The mixing in step (iv) can be carried out without requiring any shear pressure, without using rotor/stator mixing, at normal pressures, and without circulating components through a pump. It can be performed in the absence of mechanical agitation. It can be performed in the absence of thermal inversion.

The mixture of the composition and the aqueous component can be gently agitated/mixed in order to form an oil-in-water emulsion. The gentle mixing is provided by means other than homogenization, microfiltration, microfluidisation, sonication (or other high shear or high energy processes) or a phase inversion temperature process in which the temperature of the emulsion is raised until it inverts. Suitably, the gentle agitation may comprise inversion of the mixture by hand, or it may comprise stirring, or it may comprise mixing by passing through a syringe, or it may comprise any similar process. Overall, mixing is achieved by applying controlled minimal dispersion force. Inclusion of mechanical mixing components (e.g. magnetic stirring bars) is ideally avoided.

The step of combining the squalene/tocopherol/surfactant composition and aqueous component can take place below 55° C. e.g. anywhere in the range of 5-50° C., for example between 10-20° C., between 20-30° C., between 30-50° C., or between 40-50° C. The process can usefully take place at room temperature i.e. about 20-25° C. This step is ideally performed at below 30° C. e.g. in the range of 15-29° C. The composition and/or the aqueous phase are preferably equilibrated to the desired temperature before being mixed. For instance, the two components could be equilibrated to 40° C. and then be mixed. After mixing, the mixture can be maintained at a temperature below 55° C. while the emulsion forms. Preferably, the squalene/tocopherol/surfactant composition and/or aqueous component are heated before mixing and held at the desired temperature (below 55° C.) until the mixing of the two components is complete and thereafter the temperature is reduced.

The squalene/tocopherol/surfactant composition is mixed with a volume excess of the aqueous component, to ensure that an oil-in-water emulsion is formed (rather than a water-in-oil emulsion). As mentioned above, the aqueous component is preferably substantially free from surfactant(s) and/or oil(s). The process suitably uses the aqueous component at a volume excess of at least 4-fold to the squalene/tocopherol/surfactant composition e.g. between 4-fold to 50-fold greater volume. Suitably the aqueous component has a volume which is 4× to 40× larger than the volume of the squalene/tocopherol/surfactant composition. More suitably the aqueous component has a volume which is from 4× to 24×, thus giving a 5-fold to 25-fold dilution. A 8× to 20× excess, such as 9× to 19× can be particularly useful, thus giving a dilution of approximately 10 to 20 fold.

In some embodiments the squalene/tocopherol/surfactant composition is mixed with a volume excess of the aqueous component of around 7× to 10×, giving a dilution of 8 to 11-fold, such as 10-fold. The emulsion may then by further diluted (e.g. around 2-fold, such as 2-fold) at a later stage by mixing with a further aqueous component, e.g. one comprising an antigen or antigenic component to form an emulsion adjuvanted vaccine. The emulsion adjuvanted vaccine therefore may contain the squalene/tocopherol/surfactant composition in around a 18 to 22-fold, such as 20-fold dilution.

Alternatively, a squalene/tocopherol/surfactant composition is mixed with a volume excess of the aqueous component of around 17× to 21×, giving a dilution of 18 to 22-fold, such as 20-fold. The emulsion may then be mixed with a dried antigen or antigenic component to form an emulsion adjuvanted vaccine. The emulsion adjuvanted vaccine therefore may contain the squalene/tocopherol/surfactant composition in around a 18 to 22-fold, such as 20-fold dilution.

The skilled person can appreciate that other approaches may be taken to the presentation of initial and final emulsions depending on the manner in which antigen(s) or antigenic component(s) are provided. However, typically a final emulsion for administration will contain the squalene/tocopherol/surfactant composition in around a 18 to 22-fold, such as 20-fold dilution.

Emulsions of the invention may comprise at least 80% aqueous phase (e.g. water) v/v, such as at least 85% or at least 90%. Emulsions of the invention will typically comprise 99% aqueous phase (e.g. water) or less v/v, such as 98% or less.

The methods can be used at a lab or benchtop scale or at industrial scale. Thus the composition and/or aqueous phase (e.g. the composition) may have a volume in the range of 1-100 mL, in the range of 100-1000 mL, in the range of 1-10 L, or even in the range of 10-100 L.

The method may further comprise the step of subjecting the oil-in-water emulsion to sterilisation, such as filter sterilisation. The filter sterilisation can take place at any suitable stage e.g. when placing the emulsion into containers (the fill stage), or prior to any optional drying (which can be performed aseptically, to maintain sterility during and after drying).

### Oil-In-Water Emulsions

The invention provides oil-in-water emulsions obtainable by the method disclosed above. The oil particles in these emulsions have an average diameter of 200 nm or less, and in some embodiments within the range of 50 to 200 nm or even 100 to 200 nm, making them useful as immunological adjuvants. The particle diameter may be 50 nm or more, such as 100 nm or more in particular 125 m or more. The particle diameter may be 175 nm or less. In general, diameters above 100 nm, but less than 200 nm, are preferred, especially those of 125 to 175 nm, such as 150 to 175 nm.

The average diameter of oil particles in an emulsion can be determined in various ways e.g. using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See Light Scattering from Polymer Solutions and Nanoparticle Dispersions (W. Schartl), 2007. Dynamic light scattering (DLS) is the preferred method by which oil particle diameters are determined. The preferred method for defining the average oil particle diameter is a Z-average i.e. the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to an average diameter should be taken as an intensity-weighted average, and ideally the Z-average.

Droplets within emulsions of the invention preferably have a polydispersity index of 0.5 or less. Polydispersity is a measure of the width of the size distribution of particles and is conventionally expressed as the polydispersity index (Pdl). A polydispersity index of greater than 0.7 indicates that the sample has a very broad size distribution and a reported value of 0 means that size variation is absent, although values smaller than 0.05 are rarely seen. It is preferred for oil droplets within an emulsion of the invention to be of a relatively uniform size. Thus oil droplets in emulsions preferably have a Pdl of 0.5 or less e.g. 0.4 or less, such as 0.3 or less, in particular 0.2 or less. Pdl values are easily provided by the same instrumentation which measures average diameter.

### Downstream Processing

Oil-in-water emulsions of the invention can be filtered. This filtration removes any large oil droplets from the emulsion. Although small in number terms, these oil droplets can be relatively large in volume terms and they can act as nucleation sites for aggregation, leading to emulsion degradation during storage. Moreover, this filtration step can achieve filter sterilisation.

The particular filtration membrane suitable for filter sterilisation depends on the fluid characteristics of the oil-in-water emulsion and the degree of filtration required. A filter's characteristics can affect its suitability for filtration of the emulsion. For example, its pore size and surface characteristics can be important, particularly when filtering a squalene-based emulsion. Details of suitable filtration techniques are available e.g. in WO2011/067669.

The pore size of membranes used with the invention should permit passage of the desired droplets while retaining the unwanted droplets. For example, it should retain droplets that have a size of ≥1 um while permitting passage of droplets <200 nm. A 0.2 um or 0.22 um filter is generally ideal and can also achieve filter sterilisation.

The emulsion may be prefiltered e.g. through a 0.45 um filter. The prefiltration and filtration can be achieved in one step by the use of known double-layer filters that include a first membrane layer with larger pores and a second membrane layer with smaller pores. Double-layer filters are particularly useful with the invention. The first layer ideally has a pore size >0.3 um, such as between 0.3-2 um or between 0.3-1 um, or between 0.4-0.8 um, or between 0.5-0.7 um. A pore size of ≤0.75 um in the first layer is preferred. Thus the first layer may have a pore size of 0.6 um or 0.45 um, for example. The second layer ideally has a pore size which is less than 75% of (and ideally less than half of) the first layer's pore size, such as between 25-70% or between 25-49% of the first layer's pore size e.g. between 30-45%, such as ⅓ or 4/9, of the first layer's pore size. Thus the second layer may have a pore size <0.3 um, such as between 0.15-0.28 um or between 0.18-0.24 um e.g. a 0.2 um or 0.22 um pore size second layer. In one example, the first membrane layer with larger pores provides a 0.45 um filter, while the second membrane layer with smaller pores provides a 0.22 um filter.

The filtration membrane and/or the prefiltration membrane may be asymmetric. An asymmetric membrane is one in which the pore size varies from one side of the membrane to the other e.g. in which the pore size is larger at the entrance face than at the exit face. One side of the asymmetric membrane may be referred to as the “coarse pored surface”, while the other side of the asymmetric membrane may be referred to as the “fine pored surface”. In a double-layer filter, one or (ideally) both layers may be asymmetric.

The filtration membrane may be porous or homogeneous. A homogeneous membrane is usually a dense film ranging from 10 to 200 um. A porous membrane has a porous structure. In one embodiment, the filtration membrane is porous. In a double-layer filter, both layers may be porous, both layers may be homogenous, or there may be one porous and one homogenous layer. A preferred double-layer filter is one in which both layers are porous.

In one embodiment, the oil-in-water emulsions of the invention are prefiltered through an asymmetric, hydrophilic porous membrane and then filtered through another asymmetric hydrophilic porous membrane having smaller pores than the prefiltration membrane. This can use a double-layer filter.

The filter membrane(s) may be sterilised (e.g. autoclaved) prior to use to ensure that it is sterile.

Filtration membranes are typically made of polymeric support materials such as PTFE (poly-tetra-fluoro-ethylene), PES (polyethersulfone), PVP (polyvinyl pyrrolidone), PVDF (polyvinylidene fluoride), nylons (polyamides), PP (polypropylene), celluloses (including cellulose esters), PEEK (polyetheretherketone), nitrocellulose, etc. These have varying characteristics, with some supports being intrinsically hydrophobic (e.g. PTFE) and others being intrinsically hydrophilic (e.g. cellulose acetates). However, these intrinsic characteristics can be modified by treating the membrane surface. For instance, it is known to prepare hydrophilized or hydrophobized membranes by treating them with other materials (such as other polymers, graphite, silicone, etc.) to coat the membrane surface e.g. see section 2.1 of WO90/04609. In a double-layer filter the two membranes can be made of different materials or (ideally) of the same material.

During filtration, the emulsion may be maintained at a temperature of 40° C. or less, e.g. 30° C. or less, to facilitate successful sterile filtration. Some emulsions may not pass through a sterile filter when they are at a temperature of greater than 40° C.

It is advantageous to carry out the filtration step within 24 hours, e.g. within 18 hours, within 12 hours, within 6 hours, within 2 hours, within 30 minutes, of producing the emulsion because after this time it may not be possible to pass the emulsion through the sterile filter without clogging the filter, as discussed in Lidgate, 1992.

Methods of the invention may be used at large scale. Thus a method may involve filtering a volume greater than 1 liter e.g. ≥5 liters, ≥10 liters, ≥20 liters, ≥50 liters, ≥100 liters, ≥250 liters, etc.

In some embodiments an emulsion which has been prepared according to the invention can be subjected to microfluidisation. Thus, for instance, the invention can be used prior to microfluidisation to reduce the degree of microfluidising which is required for giving a desired result. Thus, if desired, microfluidisation can be used but the overall shear forces imparted on the emulsion can be reduced.

Oil-in-water emulsions of the invention can be dried (optionally after being filtered, as discussed above). Drying can conveniently be achieved by lyophilisation, but other techniques can also be used e.g. spray drying. These dried emulsions can be mixed with an aqueous component to provide once again an emulsion of the invention. Thus the invention provides a dry material (e.g. a lyophilisate) which, when reconstituted with an aqueous component, provides an oil-in-water emulsion of the invention.

As used herein, lyophilisation refers to the process of removing water from a frozen sample by sublimation and desorption under vacuum. Lyophilisation enables storage and use of vaccines independent of the cold chain. As lyophilisation improves the thermal stability of vaccines, it permits efficient distribution of vaccines. Storage and shipping becomes relatively easy as the bulky liquid vaccine formulations are transformed to dry cake-like forms. Lyophilisation of protein, live-attenuated or inactivated virus, or bacteria-containing vaccines is a routine practice for prolonging shelf-life and increasing resistance to thermal stress. Adjuvanted vaccines have added components that may create technical issues in successful lyophilisation. Hence cold chain storage becomes crucial to retain the stability of different components—antigen and adjuvants (as in some cases antigen and adjuvants are mixed immediately prior to administration). If antigen and adjuvant can be lyophilised in a single vial, cold chain maintenance can be avoided and the mixing of adjuvant and antigen prior to administration can be replaced by the simpler process of reconstituting lyophilised vaccine with a diluent.

As used herein, “dry material” and “dried material” refer to material which is substantially free of water or substantially free of an aqueous phase (e.g. it is substantially anhydrous). The dry material will usually take the form of a powder or a cake.

The invention also provides processes for preparing said dry material by preparing an oil-in-water emulsion according to the invention and subjecting it to a drying process. Suitably the emulsion is combined with (or already includes) one or more lyophilisation stabilizers prior to lyophilisation. The emulsion may also be combined with at least one antigen or antigenic component prior to drying, optionally in addition to one or more lyophilisation stabilizers.

A dry emulsion can be provided with other components in liquid form (e.g. an antigen or antigenic and/or an aqueous component). These components can be mixed in order to reconstitute the dry component and give a liquid composition for administration to a patient. A dried component will typically be located within a vial rather than a syringe.

A lyophilised component (e.g. the emulsion) may include lyophilisation stabilizers. These stabilizers include substances such as sugar alcohols (e.g. mannitol, etc.) or simple saccharides such as disaccharides and trisaccharides. Lyophilisation stabilizers are preferably small saccharides such as disaccharides. They preferably include saccharide monomers selected from glucose, fructose and galactose, and glucose-containing disaccharides and fructose-containing disaccharides are particularly preferred. Examples of preferred disaccharides include sucrose (containing glucose and fructose), trehalose (containing two glucose monosaccharides) and maltulose (containing glucose and fructose), more preferably sucrose. such as lactose, sucrose or mannitol, as well as mixtures thereof e.g. lactose/sucrose mixtures, sucrose/mannitol mixtures, etc.

An advantage of the oil-in-water emulsions of the invention and the methods for making them according to the invention is that when they are reconstituted with an aqueous component following drying, the resultant oil-in-water emulsion can retain its original properties from prior to drying (e.g. its average oil particle diameter).

### The Antigen or Antigenic Component

Although it is possible to administer oil-in-water emulsion adjuvants on their own to subjects (e.g. to provide an adjuvant effect for an antigen or antigenic component that has been separately administered to the patient), it is more usual to admix the adjuvant with an antigen or antigenic component prior to administration, to form an antigen or antigenic component containing composition e.g. a vaccine. Mixing of emulsion and antigen or antigenic component may take place extemporaneously, at the time of use, or can take place during vaccine manufacture, prior to filling. The emulsions of the invention can be used in either situation.

Various antigens or antigenic components can be used with oil-in-water emulsions, including but not limited to: viral antigens, such as viral surface proteins; bacterial antigens, such as protein and/or saccharide antigens; fungal antigens; parasite antigens; and tumor antigens.

Suitably the antigen comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

The antigen may be derived (such as obtained from) from a human or non-human pathogen including, e.g., bacteria, fungi, parasitic microorganisms or multicellular parasites which infect human and non-human vertebrates, or from a cancer cell or tumor cell.

In one embodiment the antigen is a recombinant protein, such as a recombinant prokaryotic protein.

In certain embodiments of the invention the antigen or antigenic component is derived from one or more influenza strains (i.e. a monovalent or multivalent, such as trivalent or quadrivalent influenza vaccine, which may be whole, split, purified or recombinant).

In certain embodiments of the invention the antigen or antigenic component is derived from cytomegalovirus (CMV), e.g. penta antigen (Chandramouli, 2017).

A solution of the antigen or antigenic component will normally be mixed with the emulsion e.g. at a 1:1 volume ratio. This mixing can either be performed by a vaccine manufacturer, prior to filling, or can be performed at the point of use, by a healthcare worker. An alternative formulation includes both antigen or antigenic component and emulsion in dried form in a single container for reconstitution.

### Uses of the Oil-In-Water Emulsions of the Invention

Oil-in-water emulsions of the invention are suitable for use as adjuvants for an antigen or antigenic component. Suitably these adjuvants are administered as part of a vaccine. Thus the invention provides an antigen or antigenic composition, such as a vaccine, comprising (i) an oil-in-water emulsion of the invention, and (ii) an antigen or antigenic component. These can be made by mixing an oil-in-water emulsion of the invention with an antigen or antigenic component.

The invention also provides kits comprising: an oil-in-water emulsion of the invention; and an antigen or antigenic component. The invention also provides kits comprising: a squalene/tocopherol/surfactant composition; an aqueous component; and an antigen or antigenic component. Mixing of the kit components provides a vaccine formulation of the invention.

The invention also provides kits comprising a squalene/tocopherol/surfactant composition of the invention and an aqueous component, either or both of which includes an antigen or antigenic component. Mixing of the kit components provides a vaccine formulation of the invention.

Although it is possible to administer oil-in-water emulsion adjuvants on their own to patients (e.g. to provide an adjuvant effect for an immunogen that has been separately administered), it is more usual to admix the adjuvant with an antigen or antigenic component prior to administration, to form an antigen or antigenic composition e.g. a vaccine. Mixing of emulsion and antigen or antigenic component may take place extemporaneously, at the time of use, or can take place during vaccine manufacture, prior to filling.

Overall, therefore, the invention can be used when preparing mixed vaccines or when preparing kits for mixing as discussed above. Where mixing takes place during manufacture then the volumes of bulk antigen or antigenic component and emulsion that are mixed will typically be greater than 1 liter e.g. ≥5 liters, ≥10 liters, ≥20 liters, ≥50 liters, ≥100 liters, ≥250 liters, etc.

Where mixing takes place at the point of use then the volumes that are mixed will typically be smaller than 1 milliliter e.g. ≤0.6 ml, ≤0.5 ml, ≤0.4 ml, ≤0.3 ml, ≤0.2 ml, etc. In both cases it is usual for substantially equal volumes of emulsion and antigen or antigenic solution to be mixed i.e. substantially 1:1 (e.g. between 1.1:1 and 1:1.1, preferably between 1.05:1 and 1:1.05, and more preferably between 1.025:1 and 1:1.025). In some embodiments, however, an excess of emulsion or an excess of antigen or antigenic may be used (WO2007/052155). Where an excess volume of one component is used, the excess will generally be at least 1.5:1 e.g. ≥2:1, ≥2.5:1, ≥3:1, ≥4:1, ≥5:1, etc.

Where an antigen or antigenic component and an adjuvant are presented as separate components within a kit, they are physically separate from each other within the kit, and this separation can be achieved in various ways. For instance, the components may be in separate containers, such as vials. The contents of two vials can then be mixed when needed e.g. by removing the contents of one vial and adding them to the other vial, or by separately removing the contents of both vials and mixing them in a third container.

In another arrangement, one of the kit components is in a syringe and the other is in a container such as a vial. The syringe can be used (e.g. with a needle) to insert its contents into the vial for mixing, and the mixture can then be withdrawn into the syringe. The mixed contents of the syringe can then be administered to a patient, typically through a new sterile needle. Packing one component in a syringe eliminates the need for using a separate syringe for patient administration.

In another useful arrangement, the two kit components are held together but separately in the same syringe e.g. a dual-chamber syringe. When the syringe is actuated (e.g. during administration to a patient) then the contents of the two chambers are mixed. This arrangement avoids the need for a separate mixing step at time of use.

The contents of the various kit components can all be in liquid form, but in some embodiments a dry emulsion might be included.

Vaccines are typically administered by injection, particularly intramuscular injection. Compositions of the invention are generally presented at the time of use as aqueous emulsions and are ideally suitable for intramuscular injection. In some embodiments of the invention the compositions are in aqueous form from the packaging stage to the administration stage. In other embodiments, one or more components of the compositions may be packaged in dried (e.g. lyophilised) form, and an adjuvant for actual administration may be reconstituted when necessary. The emulsion may thus be distributed as a lyophilized cake, as discussed above.

One possible arrangement according to a preferred aspect of the present invention comprises a dried emulsion component in a vial and an antigen or antigenic component and/or aqueous component in a pre-filled syringe.

The present invention also provides an arrangement comprising a dried emulsion of the present invention and a separate liquid antigen or antigenic component.

Also provided by the present invention is a dried cake formed from the emulsion of the invention. The cake may be provided in combination with a separate aqueous phase. The arrangement may further comprise an antigen or antigenic component which may be in liquid or dried form.

The present invention also provides a dried mixture wherein the mixture comprises the emulsion of the present invention in combination with an antigen or antigenic component. Preferably the mixture is a lyophilised mixture. Reconstitution of this mixture with an aqueous component provides an antigen or antigenic composition of the invention.

The invention also provides a kit for preparing an oil-in-water emulsion of the invention, wherein the kit comprises an oil-in-water emulsion of the invention in dry form and an aqueous phase in liquid form. The kit may comprise two vials (one containing the dry emulsion and one containing the aqueous phase) or it may comprise one ready filled syringe and one vial e.g. with the contents of the syringe (the aqueous component) being used to reconstitute the contents of the vial (the dry emulsion) prior to administration to a subject. In other embodiments of the invention the oil-in-water emulsion in dry form is combined with an antigen or antigenic component that is also in dry form.

If vaccines contain components in addition to emulsion and antigen or antigenic component, then these further components may be included in one of the two kit components according to embodiments of the invention or may be part of a third kit component.

Suitable containers for mixed vaccines of the invention, or for individual kit components, include vials and disposable syringes. These containers should be sterile.

Where a composition/component is located in a vial, the vial is preferably made of a glass or plastic material. The vial is preferably sterilized before the composition is added to it. To avoid problems with latex-sensitive patients, vials are preferably sealed with a latex-free stopper, and the absence of latex in all packaging material is preferred. In one embodiment, a vial has a butyl rubber stopper. The vial may include a single dose of vaccine/component, or it may include more than one dose (a ‘multidose’ vial) e.g. 10 doses. In one embodiment, a vial includes 10×0.25 ml doses of emulsion. Preferred vials are made of colourless glass.

A vial can have a cap (e.g. a Luer lock) adapted such that a pre-filled syringe can be inserted into the cap, the contents of the syringe can be expelled into the vial (e.g. to reconstitute dried material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed.

Where a composition/component is packaged into a syringe, the syringe will not normally have a needle attached to it, although a separate needle may be supplied with the syringe for assembly and use. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of adjuvant or vaccine. The syringe will generally have a tip cap to seal the tip prior to attachment of a needle, and the tip cap is preferably made of a butyl rubber. If the syringe and needle are packaged separately then the needle is preferably fitted with a butyl rubber shield.

The emulsion may be diluted with a buffer prior to packaging into a vial or a syringe. Typical buffers include: a phosphate buffer; a Tris buffer; a borate buffer; a succinate buffer; a histidine buffer; or a citrate buffer. Dilution can reduce the concentration of the adjuvant's components while retaining their relative proportions e.g. to provide a “half-strength” adjuvant.

Containers may be marked to show a half-dose volume e.g. to facilitate delivery to children. For instance, a syringe containing a 0.5 ml dose may have a mark showing a 0.25 ml volume. Where a glass container (e.g. a syringe or a vial) is used, then it is preferred to use a container made from a borosilicate glass rather than from a soda lime glass.

Compositions made using the methods of the invention are pharmaceutically acceptable. They may include components in addition to the emulsion and the optional antigen or antigenic component.

The composition may include a preservative such as thiomersal or 2-phenoxyethanol. It is preferred, however, that the adjuvant or vaccine should be substantially free from (i.e. less than 5 ug/ml) mercurial material e.g. thiomersal-free (Banzhoff, 2000; WO02/097072). Vaccines and components containing no mercury are more preferred.

The pH of an aqueous antigen or antigenic composition will generally be between 6.0 and 9.0, and more typically between 6.0 and 8.0 e.g. between 6.5 and 7.5. A process of the invention may therefore include a step of adjusting the pH of the adjuvant or vaccine prior to packaging or drying.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements.

The compositions can be administered in various ways. The most preferred immunisation route is by intramuscular injection (e.g. into the arm or leg), but other available routes include subcutaneous injection, intranasal, oral, intradermal, transcutaneous, transdermal, etc. Compositions suitable for intramuscular injection are most preferred.

Adjuvants or vaccines prepared according to the invention may be used to treat both children and adults. The patient may be less than 1 year old, 1-5 years old, 5-15 years old, 15-55 years old, or at least 55 years old. The patient may be elderly (e.g. ≥50 years old, preferably ≥65 years), the young (e.g. ≤5 years old), hospitalised patients, healthcare workers, armed service and military personnel, pregnant women, the chronically ill, immunodeficient patients, and people travelling abroad. The vaccines are not suitable solely for these groups, and may be used more generally in a population.

Adjuvants or vaccines of the invention may be administered to patients at substantially the same time as (e.g. during the same medical consultation or visit to a healthcare professional) other vaccines.

Suitable the adjuvants and vaccine of the present invention are intended for administration to humans. A typical adult dose, for administration through routes such as intramuscular, is in the region of 250 ul to 1 ml, such as 400 to 600 ul, in particular about 500 ul.

### General

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10% of the given figure, such as x±5% of the given figure.

As used herein, the singular forms “a,” “an” and “the” include plural references unless the content clearly dictates otherwise.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encephalopathies (TSEs), and in particular free from bovine spongiform encephalopathy (BSE). Overall, it is preferred to culture cells in the total absence of animal-derived materials.

The invention is illustrated by reference to the following clauses:
Clause 1. A composition comprising squalene, a tocopherol and a biocompatible metabolisable surfactant, wherein the squalene is 40% v/v or more of the composition, the tocopherol is 25% v/v or less of the composition, the surfactant is 60% v/v or less of the composition, which when mixed with an excess volume of substantially surfactant-free aqueous material, forms an adjuvant having an average oil particle diameter of 200 nm or less.
Clause 2. The composition according to clause 1, wherein the squalene is 80% v/v or less of the composition.
Clause 3. The composition according to clause 2, wherein the squalene is 70% v/v or less of the composition.
Clause 4. The composition according to one of clauses 1 to 3, wherein the squalene is 50% v/v or more of the composition.
Clause 5. The composition according to one of clauses 1 to 4, wherein the squalene is 55 to 65% v/v of the composition.
Clause 6. The composition according to any one of clauses 1 to 5, wherein the tocopherol is 20% v/v or less of the composition.
Clause 7. The composition according to any one of clauses 1 to 6, wherein the tocopherol is 10% v/v or more of the composition.
Clause 8. The composition according to any one of clauses 1 to 7, wherein the surfactant is 50% v/v or less of the composition.
Clause 9. The composition according to clause 8, wherein the surfactant is 40% v/v or less of the composition.
Clause 10. The composition according to clause 9, wherein the surfactant is 30% v/v or less of the composition.
Clause 11. The composition according to any one of clauses 1 to 10, wherein the surfactant is 10% v/v or more of the composition.
Clause 12. The composition according to clause 11, wherein the surfactant is 20% v/v or more of the composition.
Clause 13. A composition comprising squalene, a tocopherol and a biocompatible metabolisable surfactant, wherein the squalene is 50 to 70% v/v of the composition, the tocopherol is 10 to 20% v/v of the composition and the surfactant is 10 to 40% v/v of the composition.
Clause 14. The composition according to clause 13, wherein the squalene is 55 to 65% v/v of the composition, the tocopherol is 10 to 20% v/v of the composition and the surfactant is 20 to 30% v/v of the composition.
Clause 15. The composition according to either clause 13 or 14, which when mixed with an excess volume of substantially surfactant-free aqueous material, forms an adjuvant having an average oil particle diameter of 200 nm or less.
Clause 16. A method for preparing an oil-in-water emulsion adjuvant having an average oil particle diameter of 200 nm or less and comprising squalene, a tocopherol, a biocompatible metabolisable surfactant and an aqueous component, said method comprising:
   (i) providing a composition according to any one of clauses 1 to 12 or 15;
   (ii) providing an aqueous component;
   (iii) combining the composition with an excess volume of the aqueous component to form a diluted composition; and
   (iv) mixing the diluted composition to form an oil-in-water emulsion having an average oil particle diameter of 200 nm or less.
Clause 17. A method for preparing an oil-in-water emulsion adjuvant comprising squalene, a tocopherol, a biocompatible metabolisable surfactant and an aqueous component, said method comprising mixing a composition according to any one of clauses 1 to 15 with an excess volume of an aqueous component.
Clause 18. The method of either clause 16 or 17, wherein the aqueous component includes a pH buffer.
Clause 19. The method of clause 18, wherein the aqueous component is buffered with a pH of 6 to 8.
Clause 20. The method of any one of clauses 16 to 19, wherein the aqueous component comprises an antigen or antigenic component.
Clause 21. The method of any one of clauses 16 to 20, wherein the aqueous component comprises a lyophilisation stabiliser, such as a polyol e.g. sucrose.
Clause 22. The method of any one of clauses 16 to 21, further comprising a step of sterilising the oil-in-water emulsion, such as by sterile filtration.
Clause 23. The method of any one of clauses 16 to 22, further comprising a step of drying the oil-in-water emulsion.
Clause 24. The method of clause 23, wherein the drying is by lyophilisation.
Clause 25. An oil-in-water emulsion adjuvant composition obtainable by the method of any one of clauses 16 to 22.
Clause 26. A dried composition obtainable by the method of either clause 23 or 24.
Clause 27. An oil-in-water emulsion adjuvant composition comprising squalene, a tocopherol, a biocompatible metabolisable surfactant and an excess volume of an aqueous component, wherein the squalene is 40% v/v or more of the total amount of squalene, tocopherol and surfactant, the tocopherol is 25% v/v or less of the of the total amount of squalene, tocopherol and surfactant, the surfactant is 60% v/v or less of the total amount of squalene, tocopherol and surfactant and wherein the adjuvant has an average oil particle diameter of 200 nm or less.
Clause 28. The composition according to clause 27, wherein the squalene is 80% v/v or less of the total amount of squalene, tocopherol and surfactant.
Clause 29. The composition according to clause 28, wherein the squalene is 70% v/v or less of the total amount of squalene, tocopherol and surfactant.
Clause 30. The composition according to one of clauses 27 to 29, wherein the squalene is 50% v/v or more of the total amount of squalene, tocopherol and surfactant.
Clause 31. The composition according to one of clauses 27 to 30, wherein the squalene is 55 to 65% v/v of the total amount of squalene, tocopherol and surfactant.
Clause 32. The composition according to any one of clauses 27 to 31, wherein the tocopherol is 20% v/v or less of the total amount of squalene, tocopherol and surfactant.
Clause 33. The composition according to any one of clauses 27 to 32, wherein the tocopherol is 10% v/v or more of the total amount of squalene, tocopherol and surfactant.
Clause 34. The composition according to any one of clauses 27 to 33, wherein the surfactant is 50% v/v or less of the total amount of squalene, tocopherol and surfactant.
Clause 35. The composition according to clause 34, wherein the surfactant is 40% v/v or less of the total amount of squalene, tocopherol and surfactant.
Clause 36. The composition according to clause 35, wherein the surfactant is 30% v/v or less of the total amount of squalene, tocopherol and surfactant.
Clause 37. The composition according to any one of clauses 27 to 35, wherein the surfactant is 10% v/v or more of the total amount of squalene, tocopherol and surfactant.
Clause 38. The composition according to clause 37, wherein the surfactant is 20% v/v or more of the total amount of squalene, tocopherol and surfactant.
Clause 39. An oil-in-water emulsion adjuvant composition comprising squalene, a tocopherol, a biocompatible metabolisable surfactant and an excess volume of an aqueous component, wherein the squalene is 50 to 70% v/v of the total amount of squalene, tocopherol and surfactant, the tocopherol is 10 to 20% v/v of the total amount of squalene, tocopherol and surfactant, the surfactant is 10 to 40% v/v of the total amount of squalene, tocopherol and surfactant.
Clause 40. The composition according to clause 39, wherein the squalene is 55 to 65% v/v of the composition, the tocopherol is 10 to 20% v/v of the composition and the surfactant is 20 to 30% v/v of the composition.
Clause 41. The composition according to either clause 39 or 40, having an average oil particle diameter of 200 nm or less.
Clause 42. The composition according to any one of clauses 27 to 41, comprising at least 80% v/v water, such as at least 85% or at least 90%.
Clause 43. The composition according to any one of clauses 27 to 42, comprising 99% v/v or less water, such as 98% v/v or less water.
Clause 44. A vaccine composition obtainable by the method of either clause 20 or 21.
Clause 45. A vaccine composition comprising an oil-in-water emulsion according to any one of clauses 27 to 43 and an antigen or antigenic component.
Clause 46. The composition or method according to any one of clauses 1 to 45, wherein the average oil particle diameter of the adjuvant is 50 nm or more.
Clause 47. The composition or method according to clause 46, wherein the average oil particle diameter of the adjuvant is 100 nm or more.
Clause 48. The composition or method according to clause 47, wherein the average oil particle diameter of the adjuvant is 125 nm or more.
Clause 49. The composition or method according to any one of clauses 1 to 48, wherein the average oil particle diameter of the adjuvant is 175 nm or less.
Clause 50. The composition or method according to any one of clauses 1 to 49, wherein the polydispersion index of the adjuvant is 0.5 or less.
Clause 51. The composition or method according to clause 50, wherein the polydispersion index of the adjuvant is 0.3 or less.
Clause 52. The composition or method according to clause 51, wherein the polydispersion index of the adjuvant is 0.2 or less.
Clause 53. The composition or method according to any one of clauses 1 to 52, wherein the tocopherol is alpha-tocopherol.
Clause 54. The composition or method according to any one of clauses 1 to 53, wherein the surfactant component comprises two or more surfactants.
Clause 55. The composition or method according to any one of clauses 1 to 54, wherein the surfactant component consists essentially of (such as consists of) two surfactants.
Clause 56. The composition or method according to any one of clauses 1 to 54, wherein the surfactant component consists essentially of (such as consists of) one surfactant.
Clause 57. The composition or method according to any one of clauses 1 to 56, wherein the surfactant component has an HLB of 10 to 18, such as 12 to 17, especially 13 to 16.
Clause 58. The composition or method according to any one of clauses 1 to 57, wherein the surfactant comprises polysorbates, sorbitan esters, poloxamers and/or alpha-tocopherol PEG sugar esters.
Clause 59. The composition or method according to clause 58, wherein the surfactant comprises polysorbate 20, polysorbate 80, sorbitan trioleate, sorbitan monooleate, sorbitan monolaurate, poloxamer 407, poloxamer 188 and/or TPGS.
Clause 60. The composition or method according to clause 59, wherein the surfactant comprises polysorbate 80.
Clause 61. The composition according to any one of clauses 1 to 15, 26 or 46 to 60, consisting essentially of squalene, tocopherol and biocompatible metabolisable surfactant.
Clause 62. The composition according to any one of clauses 25, 27 to 43 or 46 to 60, consisting essentially of squalene, tocopherol, biocompatible metabolisable surfactant and water.
Clause 63. The vaccine composition according to any one of clauses 44 to 60, consisting essentially of squalene, tocopherol, biocompatible metabolisable surfactant, water and antigen or antigenic component.
Clause 64. The dried vaccine composition according to clause 26, consisting essentially of squalene, tocopherol, biocompatible metabolisable surfactant and antigen or antigenic component.
Clause 65. A vaccine kit comprising:
   (i) an antigen or antigenic component; an aqueous component and a composition according to any one of clauses 1 to 15, 26, 46 to 61;
   (ii) an antigen or antigenic component; and an oil-in-water emulsion adjuvant composition according to any one of clauses 25, 27 to 43, 46 to 60 or 62;
   (iii) an aqueous component and a composition according to any one of clauses 1 to 15, 26, 46 to 61, wherein either or both of the aqueous component and the composition comprise an antigen or antigenic component; or
   (iv) an aqueous component and a dried vaccine composition according to either clause 26 or 64.
Clause 66. The composition, method or kit according to any one of clauses 1 to 65, wherein the antigen or antigenic component is derived (such as obtained from) from a human or non-human pathogen including, e.g., bacteria, fungi, parasitic microorganisms or multicellular parasites which infect human and non-human vertebrates, or from a cancer cell or tumor cell.
Clause 67. The composition, method or kit according to clause 66, wherein the antigen or antigenic component is derived from influenza.
Clause 68. The composition, method or kit according to clause 66, wherein the antigen or antigenic component is derived from CMV.

### EXAMPLES

### Example 1—Formation of Oil-In-Water Emulsions and Measurement of the Average Particle Size

Mixtures of squalene, alpha-tocopherol and polysorbate 80 in various proportions were investigated for their ability to spontaneously form small droplet emulsions on mixing with water.

### Method

### Emulsion Preparation

Appropriate proportions of squalene, D/L-alpha-tocopherol and polysorbate 80 with the percentage compositions set out in Tables 1 were stirred overnight at room temperature.

Dulbecco's Phosphate buffered saline (DPBS) was adjusted to pH 6.65-6.95. The DPBS was then warmed to around 35-40° C. before adding the oil phase (9:1 v/v ratio DPBS:oil). The mixture was maintained around 40° C. for around 1 hour, with periodic inversion of the container.

### Sizing

Emulsions were typically diluted 100× (990 uL water+10 uL emulsion) and then further diluted 5 times (400 uL water+100× diluted emulsion) to obtain a 500× dilution. Size was measured using a Malvern Zetasizer. Dilutions may be adapted as necessary if the observed kilo counts per second is too low.

### Results

The results are displayed in Table 1. Lower amounts of tocopherol in the initial mixture (10-15% v/v) resulted in emulsion droplet size less of 200 nm or less and Pdl of 0.3 or less. Generally, emulsions with higher tocopherol content and an emulsion with a composition similar to AS03 showed high droplet size and Pdl values.

Emulsion compositions with increased surfactant content displayed reduced particle size and lower Pdl. Increased particle size and lower Pdl could be achieved by increasing the squalene concentration and reducing the surfactant concentration.

A number of formulations were prepared multiple times, with no notable discrepancies in size or Pdl observed between batches, demonstrating the robustness of this method.

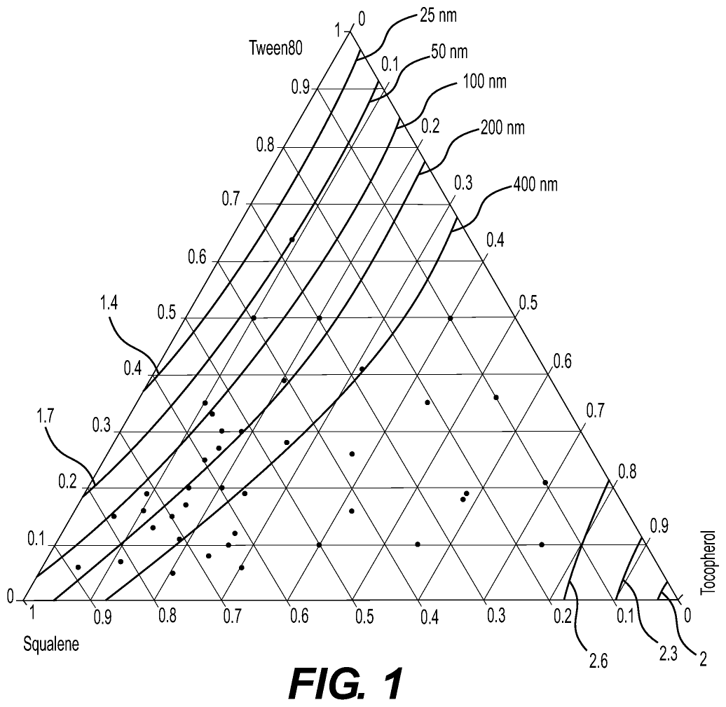

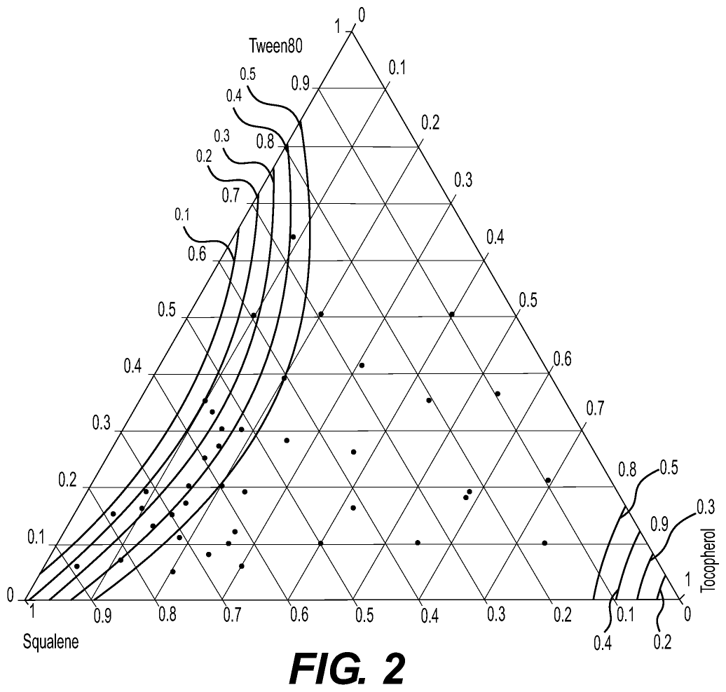

Data have been plotted in FIGS. 1 and 2, along with contour maps for particle size and polydispersity index (Pdl). Contour maps were prepared using JMP12 software using a fit model.

TABLE 1
Tocopherol-containing oil-in-water emulsion
compositions and particle size/Pdl

| | Tween80 | Squalene | Tocopherol | Size | |
|---|---|---|---|---|---|
| Formulation | (% vol.) | (% vol.) | (% vol) | (nm) | Pdl |
| 1 | 10 | 16 | 74 | 474.07 | 0.748 |
| 2 | 10 | 35 | 55 | 1363.43 | 0.902 |
| 3 | 10 | 50 | 40 | 656.60 | 0.750 |
| 4 | 10 | 64 | 26 | 315.17 | 0.642 |
| 5 | 16 | 42 | 42 | 702.83 | 0.784 |
| 6 | 16 | 74 | 10 | 170.60 | 0.208 |
| 7 | 18 | 24 | 58 | 1642.67 | 0.860 |
| 8 | 19 | 57 | 24 | 517.67 | 0.647 |
| 9 | 19 | 23 | 58 | 474.07 | 0.748 |
| 10 | 21 | 10 | 69 | 1563.67 | 0.940 |
| 11 | 26 | 37 | 37 | 279.63 | 0.871 |
| 12 | 27 | 57 | 16 | 129.60 | 0.357 |
| 13 | 28 | 46 | 26 | 960.40 | 0.804 |
| 14 | 35 | 55 | 10 | 87.25 | 0.122 |
| 14 | 35 | 55 | 10 | 79.76 | 0.113 |
| 15 | 35 | 21 | 44 | 1182.37 | 0.903 |
| 16 | 36 | 10 | 54 | 234.67 | 0.619 |
| 17 | 39 | 41 | 20 | 410.30 | 0.786 |
| 18 | 41 | 28 | 31 | 761.20 | 0.851 |
| 19 | 50 | 10 | 40 | 444.73 | 0.920 |
| 20 | 50 | 30 | 20 | 375.67 | 0.569 |
| 21 | 50 | 40 | 10 | 46.19 | 0.259 |
| 22 | 15 | 70 | 15 | 130.47 | 0.326 |
| 22 | 15 | 70 | 15 | 126.00 | 0.222 |
| 22 | 15 | 70 | 15 | 129.07 | 0.212 |
| 23 | 63.6 | 27.3 | 9.1 | 31.63 | 0.402 |
| 24 | 7 | 82 | 11 | 189.20 | 0.48 |
| 25 | 12 | 62 | 26 | 644.70 | 0.73 |
| 26 | 20 | 60 | 20 | 563.23 | 0.88 |
| 27 | 8 | 68 | 24 | 548.37 | 0.54 |
| 28 | 15 | 79 | 6 | 180.63 | 0.16 |
| 29 | 5 | 75 | 20 | 932.87 | 0.73 |
| 30 | 6 | 89 | 5 | 204.03 | 0.32 |
| 31 | 17 | 67 | 16 | 154.30 | 0.53 |
| 32 | 6 | 64 | 30 | 2132.00 | 0.78 |
| 33 | 19 | 72 | 9 | 159.10 | 0.18 |
| 34 | 11 | 71 | 18 | 406.30 | 0.70 |
| 35 | 13 | 74 | 13 | 144.27 | 0.28 |
| 41 | 33 | 55 | 12 | 94.13 | 0.166 |
| 42 | 30 | 55 | 15 | 120.73 | 0.219 |
| 43 | 30 | 52 | 18 | 134.20 | 0.360 |
| 44 | 25 | 60 | 15 | 131.73 | 0.192 |
| 45 | 20 | 65 | 15 | 139.10 | 0.215 |

### Example 2—Testing of Other Surfactants

The use of alternative surfactant(s) was investigated to determine the impact on size and Pdl.

### Method

Further formulations comprising alternative surfactant components were prepared by methods analogous to those in Example 1. A range of surfactants are featured, with differing HLB values (Table 2).

TABLE 2
Surfactants used and associated HLB values

| Surfactant | HLB |
|---|---|
| Tween 80/Polysorbate 80 | 15 |
| Tween 20/Polysorbate 20 | 16.7 |
| Span85 | 1.8 |
| Span80 | 4.3 |
| Span20 | 8.6 |
| TPGS | 13-13.2 |
| Poloxamer 188 | 29 |
| Poloxamer 407 | 22 |

### Results

TABLE 3
Further tocopherol-containing oil-in-water
emulsion compositions and particle size/Pdl

| | | | Surfactant Component(s) | | | | | | |
|---|---|---|---|---|---|---|---|---|---|
| | Sq | Toco | | A % | | B % | Calc | Size | |
| Form. | (% vol) | (% vol) | Surf A | vol | Surf B | vol* | HLB | (nm) | Pdl |
| 8 | 57 | 24 | Polysorbate | 19 | — | — | 15 | 517.67 | 0.647 |
| | | | 80 | | | | | | |
| 8a | 57 | 24 | Polysorbate | 19 | TPGS | +1.9 | 14.8 | 169.20 | 0.539 |
| | | | 80 | | | | | | |
| 8b | 57 | 24 | Polysorbate | 19 | TPGS | +3.8 | 14.7 | 132.93 | 0.493 |
| | | | 80 | | | | | | |
| 26 | 60 | 20 | Polysorbate | 20 | — | — | 15 | 563.23 | 0.88 |
| | | | 80 | | | | | | |
| 26a | 60 | 20 | Polysorbate | 20 | TPGS | +2.0 | 14.8 | 100.46 | 0.375 |
| | | | 80 | | | | | | |
| 31 | 67 | 16 | Polysorbate | 17 | — | — | 15 | 154.30 | 0.53 |
| | | | 80 | | | | | | |
| 31a | 67 | 16 | Polysorbate | 17 | TPGS | +1.7 | 14.8 | 118.00 | 0.281 |
| | | | 80 | | | | | | |
| 22 | 70 | 15 | Polysorbate | 15 | — | — | 15 | 129.07 | 0.212 |
| | | | 80 | | | | | | |
| 22a | 70 | 15 | Polysorbate | 15 | TPGS | +1.5 | 14.8 | 123.80 | 0.218 |
| | | | 80 | | | | | | |
| 22b | 70 | 15 | Polysorbate | 15 | TPGS | +3.0 | 14.7 | 122.37 | 0.208 |
| | | | 80 | | | | | | |
| 6 | 74 | 10 | Polysorbate | 16 | — | — | — | 170.60 | 0.208 |
| | | | 80 | | | | | | |
| 6a | 74 | 10 | Polysorbate | 16 | TPGS | +1.6 | 14.8 | 160.87 | 0.181 |
| | | | 80 | | | | | | |
| 6b | 74 | 10 | Polysorbate | 16 | P188 | +1.6 | 16.3 | 160.93 | 0.178 |
| | | | 80 | | | | | | |
| 6c | 74 | 10 | Polysorbate | 16 | P407 | +1.6 | 15.6 | 160.47 | 0.186 |
| | | | 80 | | | | | | |
| 6d | 74 | 10 | Polysorbate | 9.7 | Span80 | 6.3 | 10.8 | 100.06 | 0.379 |
| | | | 80 | | | | | | |
| 6e | 74 | 10 | Polysorbate | 9.7 | Span85 | 6.3 | 10.8 | 151.23 | 0.251 |
| | | | 20 | | | | | | |
| 6f | 74 | 10 | Polysorbate | 9.7 | Span20 | 6.3 | 13.5 | 160.30 | 0.222 |
| | | | 20 | | | | | | |
| 24 | 82 | 11 | Polysorbate | 7 | — | — | 15 | 189.20 | 0.48 |
| | | | 80 | | | | | | |
| 36 | 82 | 11 | Polysorbate | 7 | TPGS | +2.0 | 14.5 | 145.27 | 0.268 |
| | | | 80 | | | | | | |

*For formulation 36 and all formulations a-c, the % surfactant B is in addition to 100% of squalene, tocopherol and surfactant A. For formulations d-f, surfactant B is included in a total of 100% of squalene, tocopherol, surfactant A and surfactant B.

The results in Table 3 demonstrate that a range of surfactants can be used while still maintaining the ability to spontaneously form emulsions with small particle size and good polydispersity.

### Example 3—In Vivo Testing of Oil-In-Water Emulsions for their Potential Use as Adjuvants

The oil-in-water emulsions from Examples 1 and 2 were tested in vivo, demonstrating their use as vaccine adjuvants in mice. Two studies were performed with quadrivalent H1N1 A/Singapore, A/Hong Kong (H3N2), B/Phuket and B/Brisbane influenza vaccine (QIV) at a 0.01 ug (0.04 ug total) and a 0.1 ug (0.4 ug total) dose (Table 4). Control experiments were also carried out with antigens administered without adjuvant, or antigens administered with the known emulsion adjuvants AS03 or SEA160.

A IgG sub-type ELISA was performed to evaluate overall humoral response. T-cell activation, T-cell differentiation into CD4+ and CD8+ population and the generation of cytokines from activated T cells were analysed to determine cellular responses.

### Methods

For in vivo studies, the concentrated emulsions from Example 1 and 2 were sterile filtered using 0.22 um PES membranes.

The concentrated emulsions were diluted with an equal volume of aqueous antigen to provide the final emulsion adjuvanted vaccine formulations. Fractional emulsion doses were prepared by initial 10-fold dilution of the concentrated emulsion, before being diluted with an equal volume of aqueous antigen to provide the final fractional emulsion adjuvanted vaccine formulations.

TABLE 4
Study design with QIV antigen at 0.01 ug dose

| Group | Treatment |
|---|---|
| A | 0.01 QIV |
| B | 0.01 ug QIV + formulation 36 |
| C | 0.01 ug QIV + formulation 22 |
| D | 0.01 ug QIV + 1/10^{th} formulation 36 |
| E | 0.01 ug QIV + 1/10^{th} formulation 22 |
| F | 0.01 ug QIV + AS03 |
| G | 0.01 ug QIV + 1/10^{th} AS03 |
| H | 0.1 ug QIV + SEA160 |
| I | 0.1 QIV |
| J | 0.1 ug QIV + formulation 36 |
| K | 0.1 ug QIV + formulation 22 |
| L | 0.1 QIV ug + 1/10^{th} formulation 36 |
| M | 0.1 QIV ug + 1/10^{th} formulation 22 |
| N | 0.1 ug QIV + AS03 |
| O | 0.1 ug QIV + 1/10^{th} AS03 |
| P | 0.1 ug QIV + SEA160 |

TABLE 5
Timeline of study of adjuvants

| Day | Procedure |
|---|---|
| 1 | First immunization |
| 22 | 3pw1 Bleed |
| | Second immunization |
| 36 | 2wp2 Terminal bleed and spleen harvest from |
| | 5 animals per group |
| 43 | 3wp2 Terminal bleed and spleen harvest from |
| | remaining animals |

Formulations were administered intramuscularly 25 ul per leg (total 50 ul per animal). 8 female 6-8 week old Balb/c mice were used in each test group.

### HAI Assay

Hemagglutination inhibition (HI or HAI) occurs when antibodies in the serum obtained from mice immunized with the split antigen bind to the virus/viral particles to inhibit agglutination of the erythrocytes. Serially dilutions of obtained from the mice were plated and incubated with a fixed amount of split antigen equivalent to 8HA units (obtained from the HA titer performed). After incubation, red blood cells from chicken were added and the sample incubated another 30 min to read the plate. The highest dilution of serum that prevents hemagglutination is called the HI titer of the serum. If the serum contains no antibodies that react, then hemagglutination will be observed in all wells. Likewise, if antibodies to the virus are present, hemagglutination will not be observed until the antibodies are sufficiently diluted. HA titer is defined as the reciprocal of the dilution of the last well where virus/viral particle causes agglutination of erythrocytes.

### IqG Subtype ELISA

Split antigens from all strains in the QIV were coupled with Magplex microspheres (at conc. 1*10⁶ beads/mL) of 4 different lots. 5 ug antigen per reaction was used to couple with Magplex beads and shaken on nutator for one hour and stored at 4° C. until used. Serum was 5-fold serially diluted in the assay buffer in a 96-well flat-bottom plate. Preliminary assays were performed to determine the dilution range and starting dilutions for different antigens. For unadjuvanted and diluted adjuvant groups starting dilution was 1:20 whereas for other groups it was 1:50. The antigen coupled microspheres were removed from 4° C. and equilibrated at room temperature on the nutator and diluted in assay buffer before adding 50 uL to the plate. The secondary antibody was added after incubating the plates for one hour on a shaker and washing with the assay buffer. Plates were incubated at room temperature for another one hour, washed and the beads were resuspended in assay buffer before analysis on a FlexMap 3D machine.

End-point titers were calculated at a value of 5000. The dilution factor corresponding to the endpoint titer of 5000 was extrapolated, and thus, titers for each animal in the group were calculated. The end-point titers were further analyzed in graphpad prism using one-way ANOVA and the mean of each column was compared with the mean of every other column using Tukey's test for multiple comparisons.

### ICS Staining

5 spleens per group of animals were harvested, for ICS assay, in RPMI media containing 10% FBS. A spleen from each animal was harvested and filtered through 70 um filters. Once homogenized, RBC lysis buffer was added to filter off any RBCS in the splenocyte suspension. These cell suspensions were filtered through 70 micron cell strainers (BD Biosciences, catalog no. #352350). Cells were centrifuged at 1200 RPM for 5 minutes. The pellet was re-suspended into RPMI and centrifuged again. The new pellet was re-suspended in RPMI. These cell suspensions were counted using NucleoCounter NC-3000 by ChemoMetec. After diluting splenocytes in each tube appropriately, about 2,000,000 cells per well were plated in round-bottom 96-well plate. All the wells received Anti CD28 monoclonal antibodies (mAb) (BD Biosciences #553294) to provide co-stimulatory signal needed for T-cell activation. 6 different types of treatments were given to each group:
No stimulation (with PBS): A negative control to identify the baseline responses
Stimulation by Anti-CD3 mAb (BD Biosciences #553057): Positive group which ideally shows maximal responses
A/Singapore split antigen 2.5 μg/mL protein (Protein used for immunization)
Recombinant HA protein (rHA) for A/HongKong from Protein Biosciences @2.5 μg/mL
rHA for B/Brisbane @2.5 μg/mL
rHA for B/Phuket @2.5 μg/mL

Cells were incubated overnight at 37° C. with BFA and fixed the next day using cytofix/cytoperm and then stained using to define CD4+ T cell responses. CD 107a was added in this cocktail in each well to stain the granulating T cells. The compensation controls were used to compensate overlap in signal from the fluorochromes conjugated with aforementioned surface and intracellular antibodies.

The data acquired from LSRII was analysed using FlowJo software and a specific gating strategy was applied to obtain specific CD4+, CD8+ T cell responses and cytokine positive T-cell responses.

The following cytokine combinations were used for Boolean gating:
Th1: IFNγ producing T cells only
Th2: IL4+IL13+ producing T cells only
Th17: IL17a and/or IL17f producing T cells only
Th0: TNFα and/or IL-2 producing T-cells that is negative for all other cytokines.

### Statistical Analysis

One-Way ANOVA followed by Tukey's test.

### Results

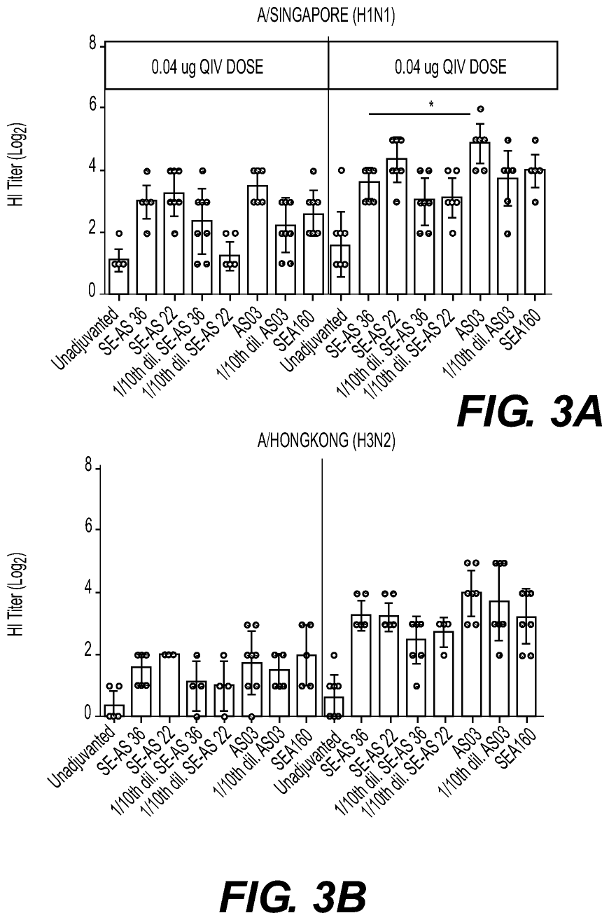

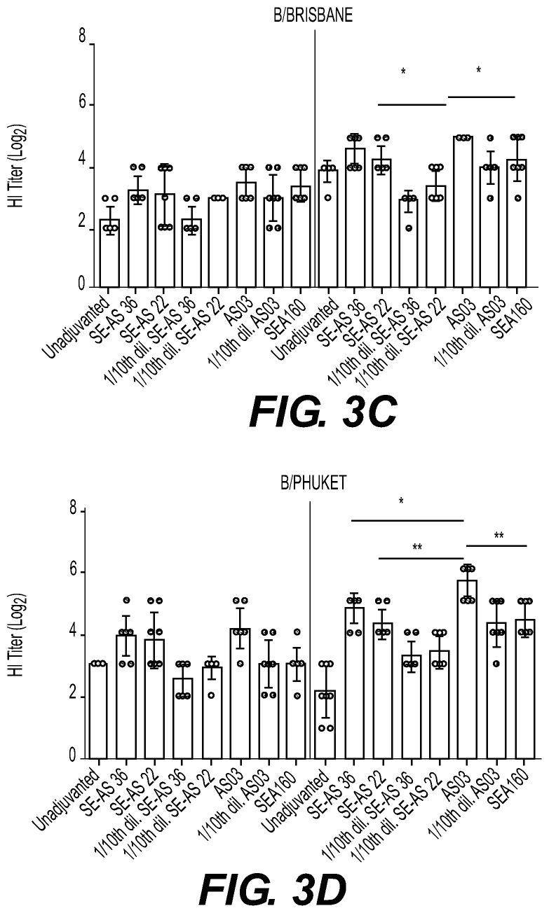

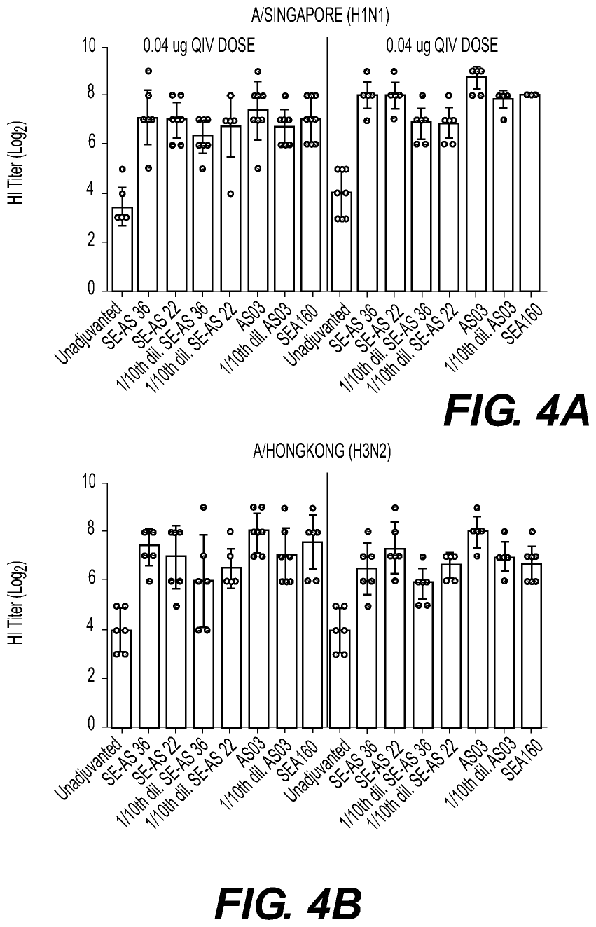

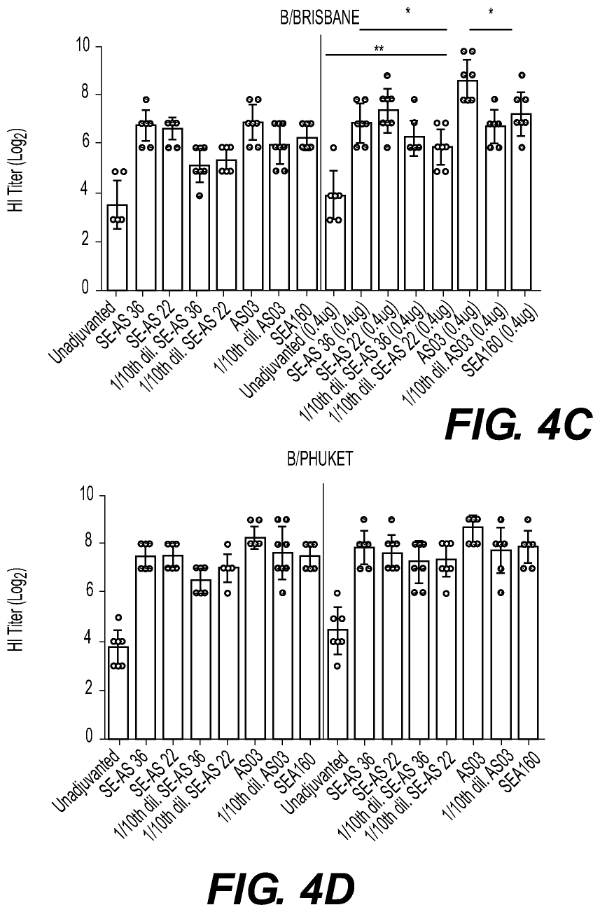

The results are presented in FIG. 3 (3 weeks post first immunisation) and FIG. 4 (3 weeks post second immunisation).

All adjuvanted mice showed HAI titers higher than the unadjuvanted groups, confirming the adjuvant activity of the novel emulsion adjuvants. Generally, across all 4 antigens, AS03, formulation 22 and formulation 36 showed significantly non-inferior responses. An exception to this is B/Brisbane, in which AS03 showed significantly higher titers compared to formulation 36 and SEA160. 1/10^{th} diluted adjuvant groups showed lower response compared to the full-dose adjuvants, however the difference was not significant. Additionally, formulations 22 and 36 showed similar responses to SEA160 (a formulation not containing tocopherol). After 2wp2, the unadjuvanted dose group maintained the significantly lower responses compared to full-dose adjuvanted groups. The trend within other adjuvanted groups remained similar.

### IqG Subtype ELISA

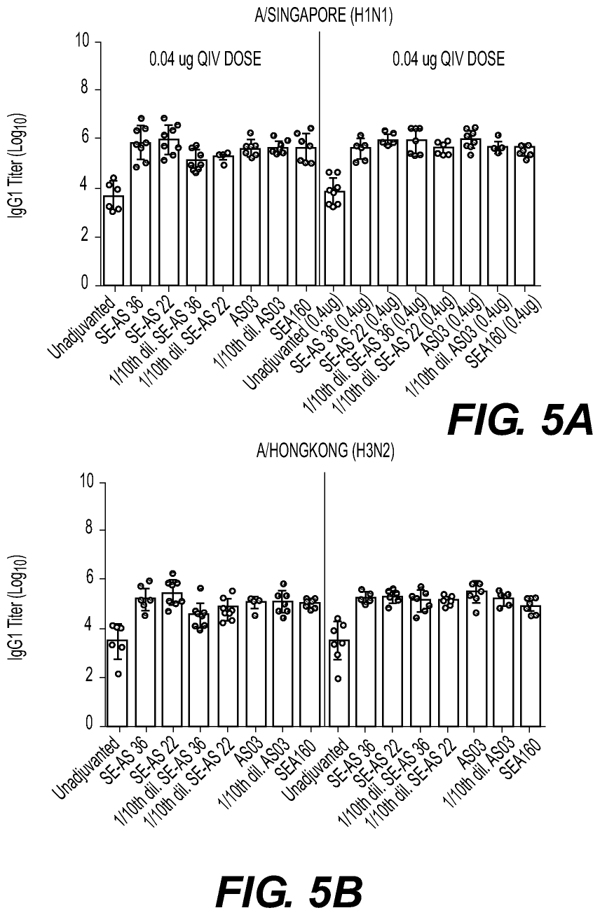

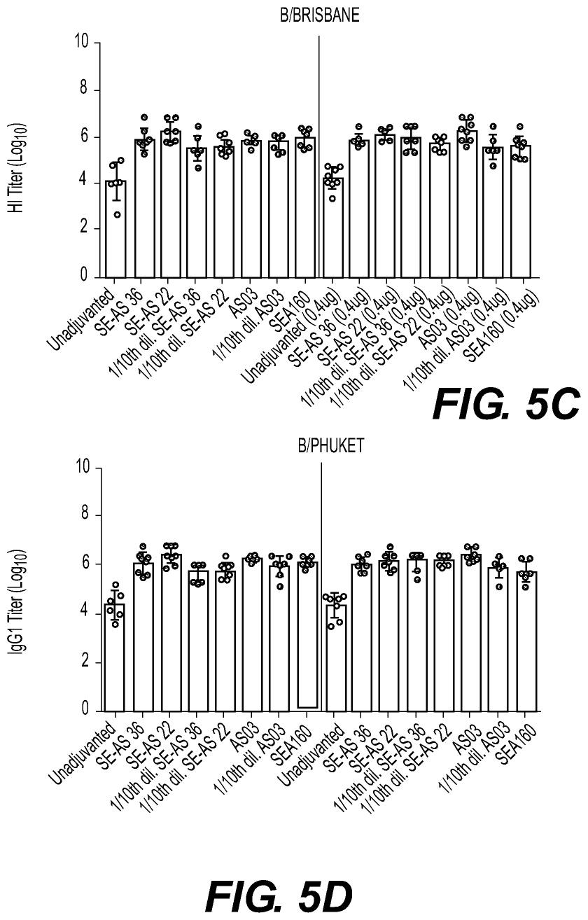

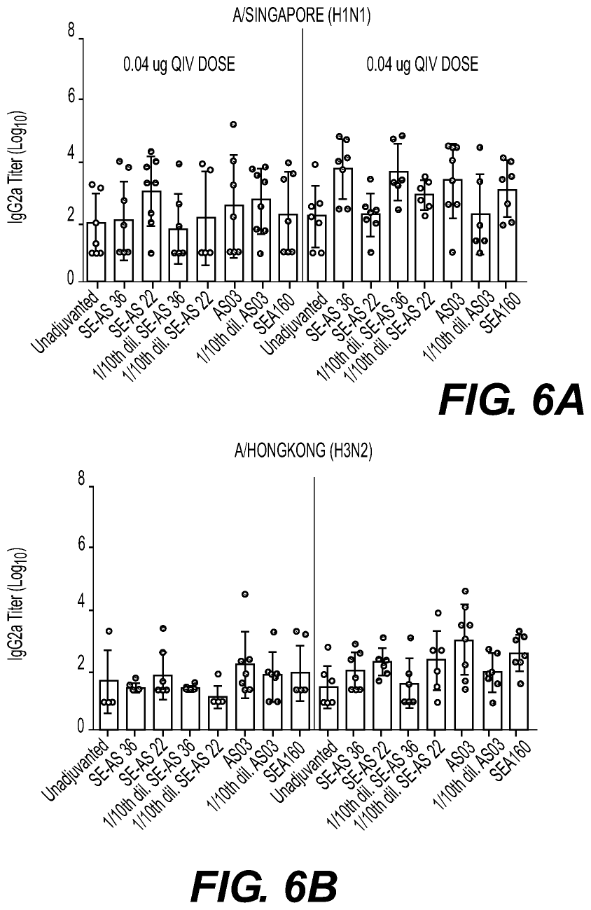

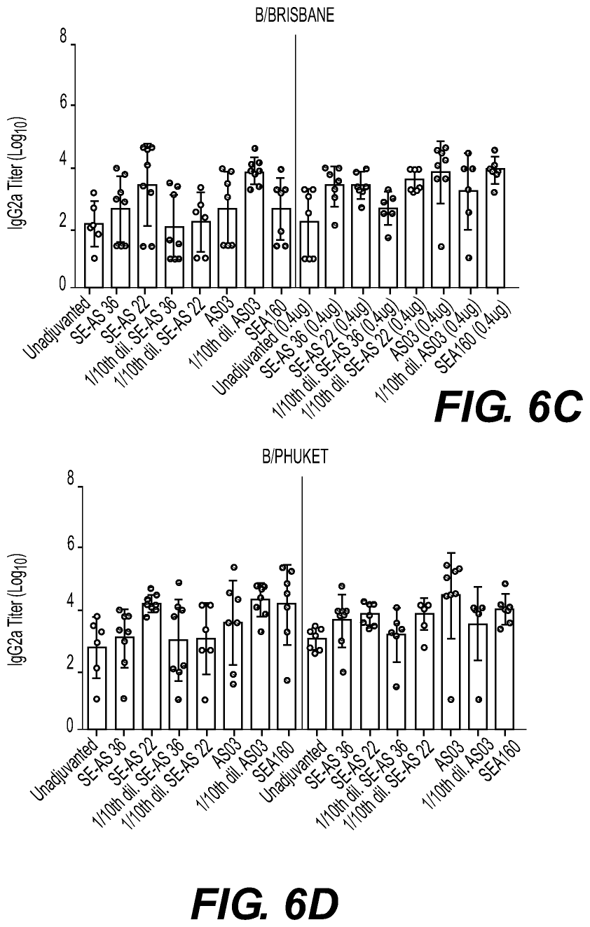

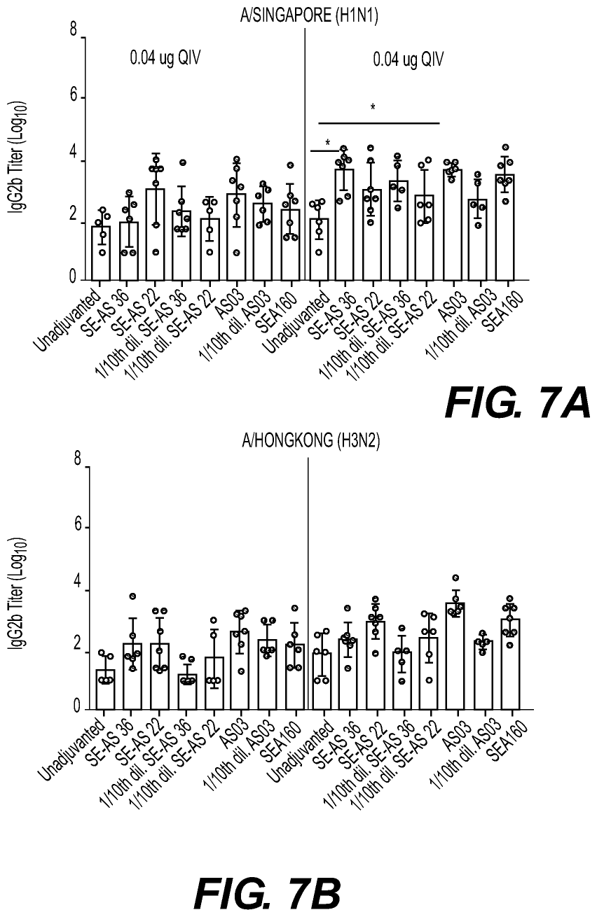

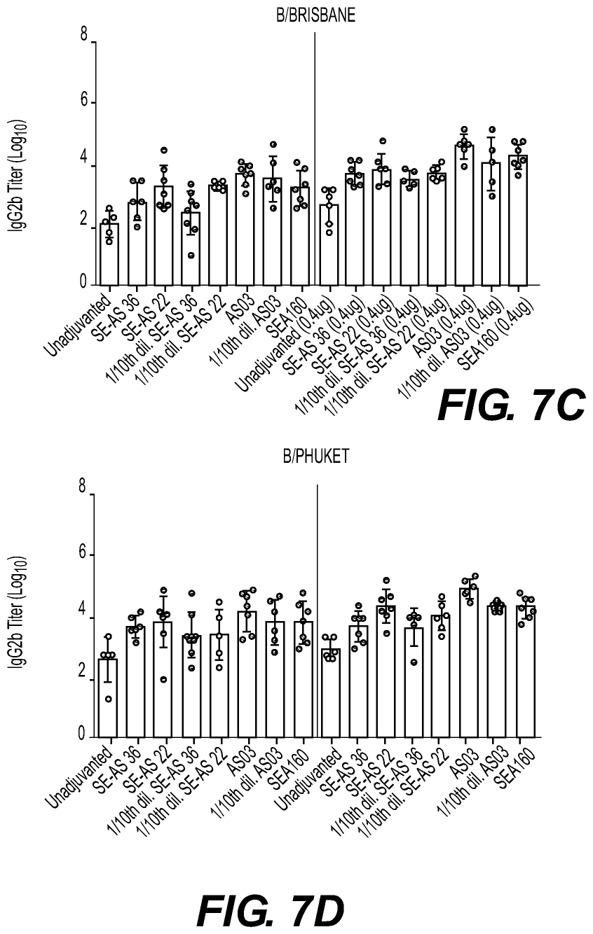

For IgG1 and IgG2a sub-type titers, all adjuvanted groups showed higher titers than the unadjuvanted groups (FIGS. 5 and 6). Across all four antigens, formulations 36, 22 and AS03 displayed significantly non-inferior response. No obvious trend was observed across groups for IgG2a and IgG2b titers. For IgG2b sub-type titers (FIG. 7), the results were similar to those observed with the HAI titers (see Example 3 and FIGS. 2 and 4). This suggests that the HAI antibodies could be of the IgG2b subtype.

### ICS Staining

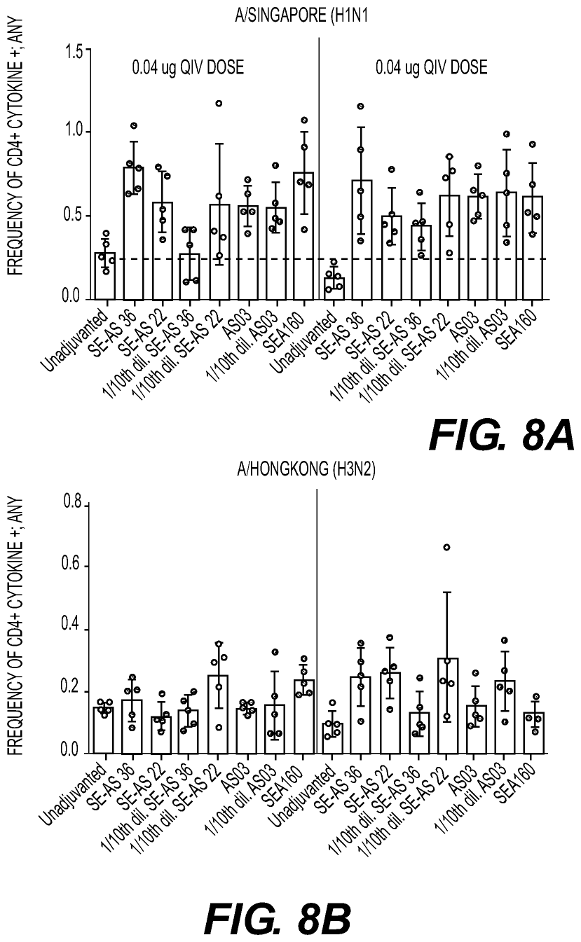

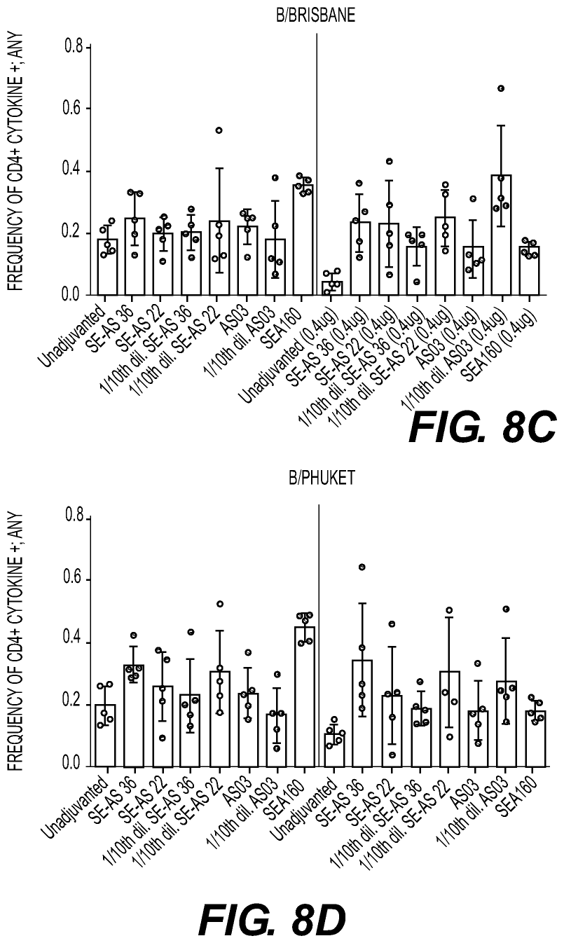

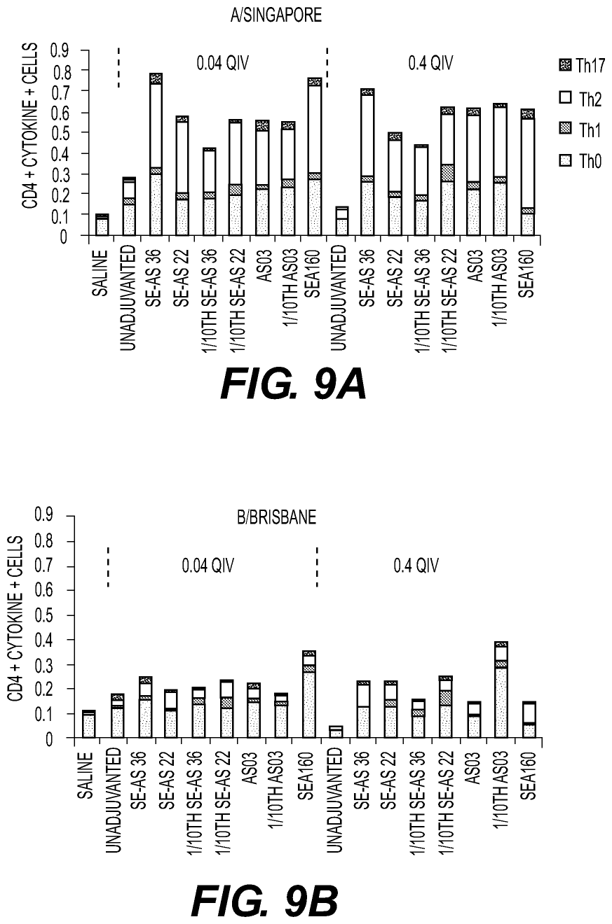

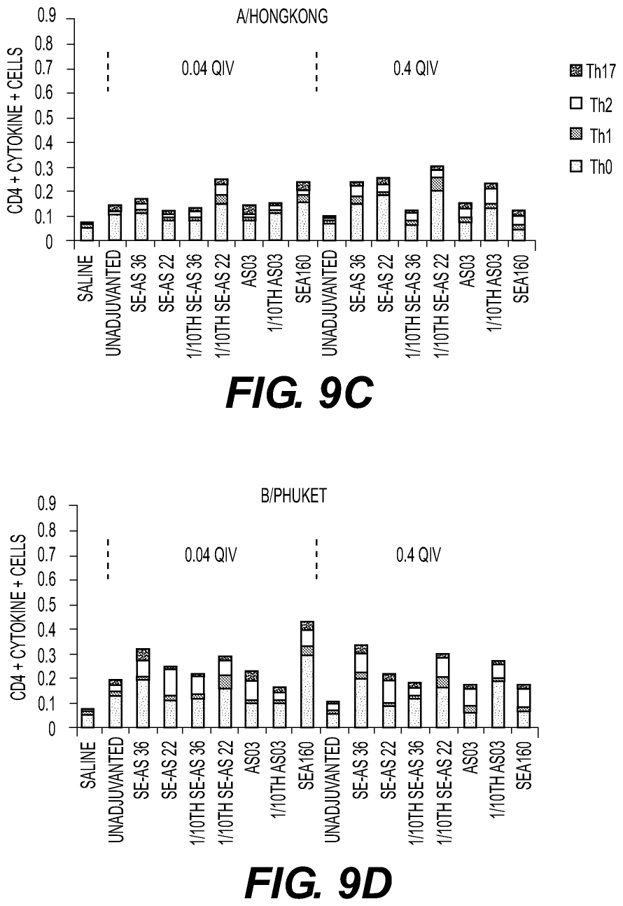

The results are presented in FIGS. 8 and 9. Splenocytes restimulated with the split antigen for A/Singapore showed an overall higher T cell response than the other strains for which rHA was used. CD8 T cell frequency was low overall across all antigens (data not shown). Overall, frequency for CD4+ T cells remained higher for adjuvanted groups at both QIV doses compared to the unadjuvanted group. Th0 and Th2 responses were higher that Th1 and Th17. The results observed corroborate with the HA data previously presented above.

### Example 4—Sterile Filterability

Formulations 22, 36, 44 and 45 were prepared and filtered through 0.22 um polyether sulfonate (PES) filter. 1 mL emulsion was drawn in a 3 mL syringe and filtered through a 33 mm, 0.22 um PES syringe filter into a vial. The emulsions were tested for size, Pdl and percentage content for squalene and tocopherol using UPLC-PDA.

### Method

Formulations 22, 36, 44 and 45 were prepared as described in Examples 1 and 2. The percentage content of squalene and tocopherol in the emulsion after emulsification was determined using Ultra-High Pressure Liquid Chromatography (UPLC). A Xterra C18 column from Waters® was used. The mobile phase was 95:5 methanol: acetonitrile. The run time was 15 mins at 1 mL/min flow rate. The column was heated at 37° C. during elution and a PDA detector was used to record the eluting peaks. The retention time for tocopherol was 4.4 mins and squalene was 7.5 mins. A standard curve of squalene and tocopherol mixture was run before each run with concentrations ranging from 600 ug/mL to 2.34 ug/mL. Using the slope and intercept from this standard curve, the concentrations of squalene and tocopherol in the emulsion samples was determined.

### Results

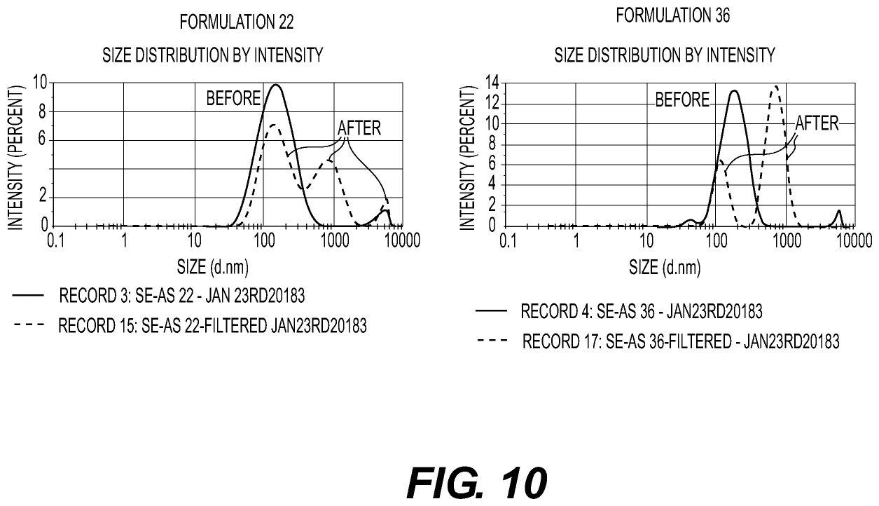

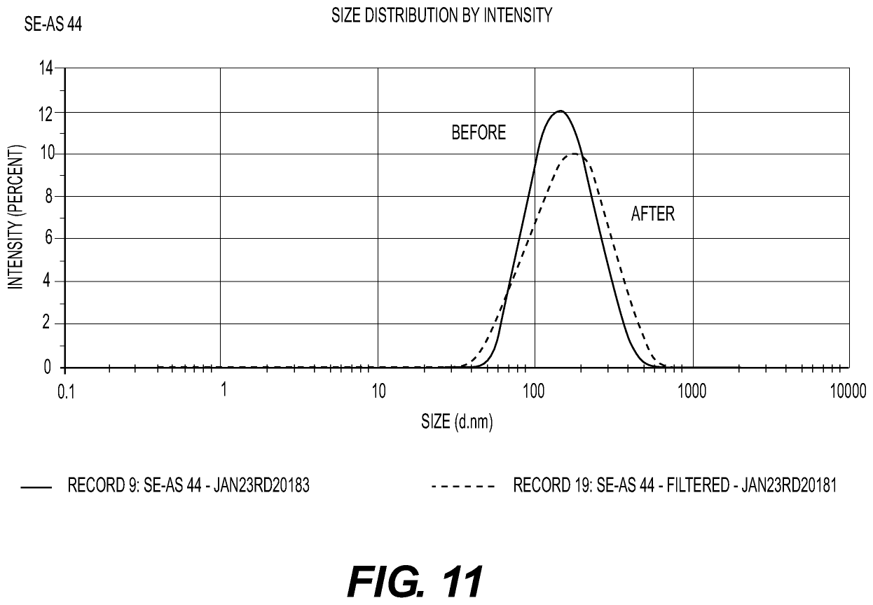

The results are shown in Table 6 and FIGS. 10 and 11. The polydispersity of formulations 22 and 36 increases after filtration showing a bimodal size distribution, accompanied by significant loss of oil content after filtration. Sterile filtration of the emulsions leads to increased particle size and Pdl outside of the required thresholds. Formulation 44 maintained its size and Pdl after filtration; formulation 45 exhibited an increase in size and Pdl. UPLC-PDA showed minimal loss in content compared to formulations 22 and 36 (Table 6). Formulation 44 maintained its particle size and Pdl post-filtration, whilst displaying minimal loss in oil content.

TABLE 6
Size and Pdl before and after filtration
and content loss after filtration.

| | Before | | After | | | |
|---|---|---|---|---|---|---|
| | filtration | | filtration | | Content loss after | |
| | Size | | Size | | filtration (%) | |
| Formulation | (nm) | Pdl | (nm) | Pdl | Squalene | Tocopherol |
| 22 | 135.2 | 0.241 | 230.1 | 0.484 | 31.12 | 32.38 |
| 36 | 173.3 | 0.283 | 371.3 | 0.619 | 45.12 | 61.02 |
| 44 | 133.7 | 0.184 | 134.9 | 0.203 | 16.30 | 12.64 |
| 45 | 141.5 | 0.201 | 177.2 | 0.329 | Not | Not |
| | | | | | measured | measured |

### Example 5—Comparison of Formulation Emulsion Stability at Elevated Temperatures

The stabilities of formulations 22, 36 and 44 at three temperature points (5° C., 25° C. and 50° C.) were evaluated over a period of 4 weeks. The pH, osmolality, particle size and Pdl of the emulsions were measured.

### Method

pH was measured using Thermo's Orion pH meter. Osmolality was measured using Model 2020 osmometer, size was measured using Malvern's Zetasizer and % content was measured using UPLC-PDA.

pH, osmolality, size and Pdl were measured up to 10 weeks.

### Results

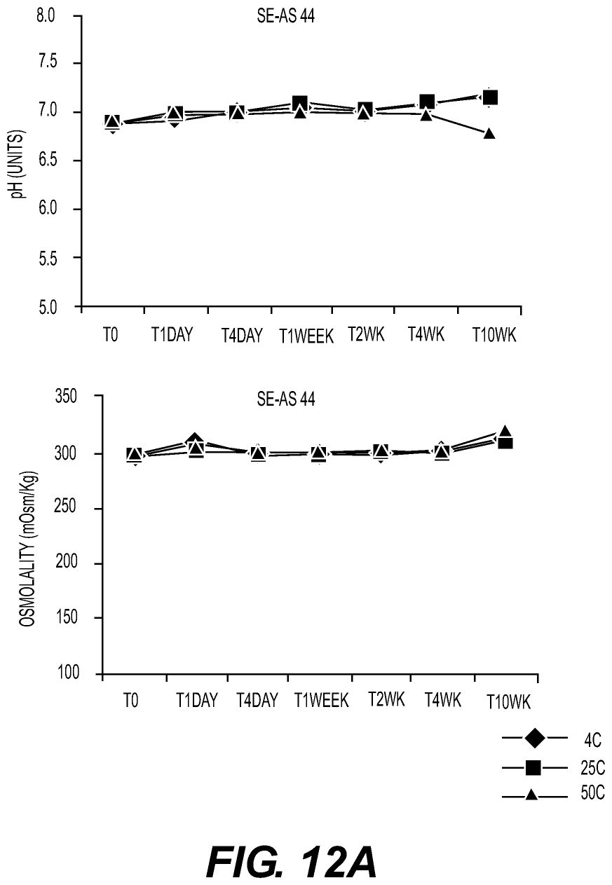

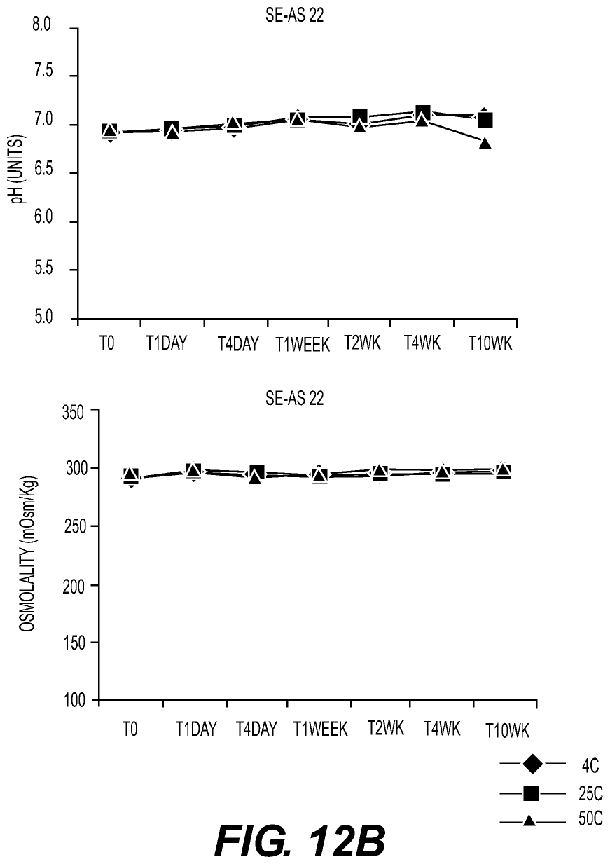

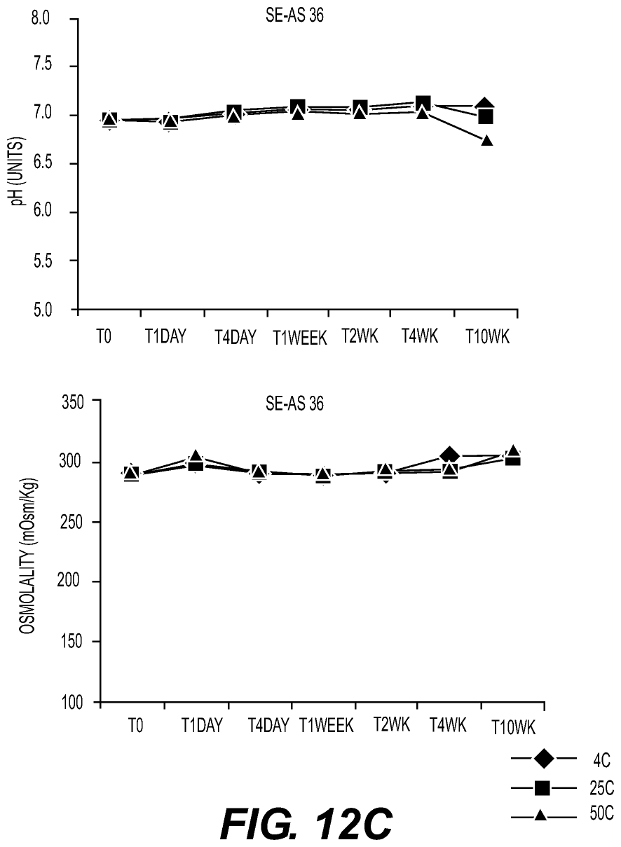

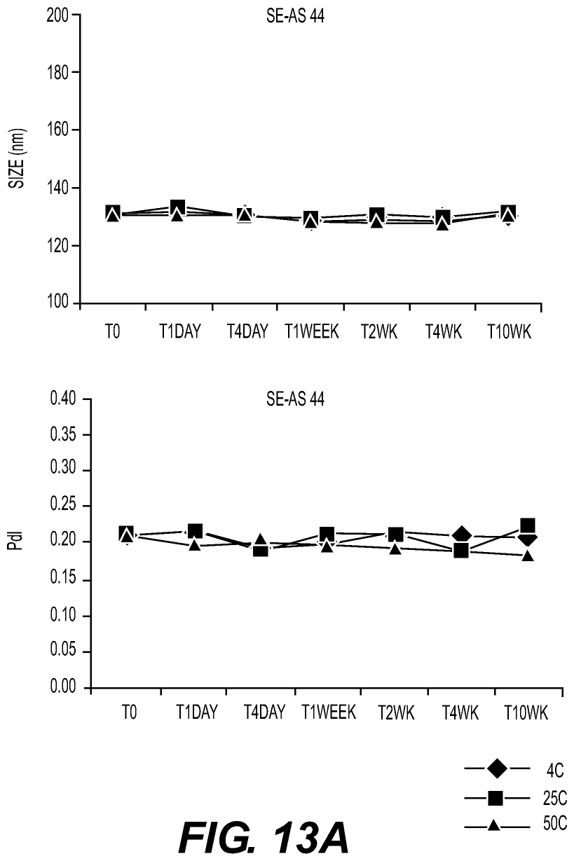

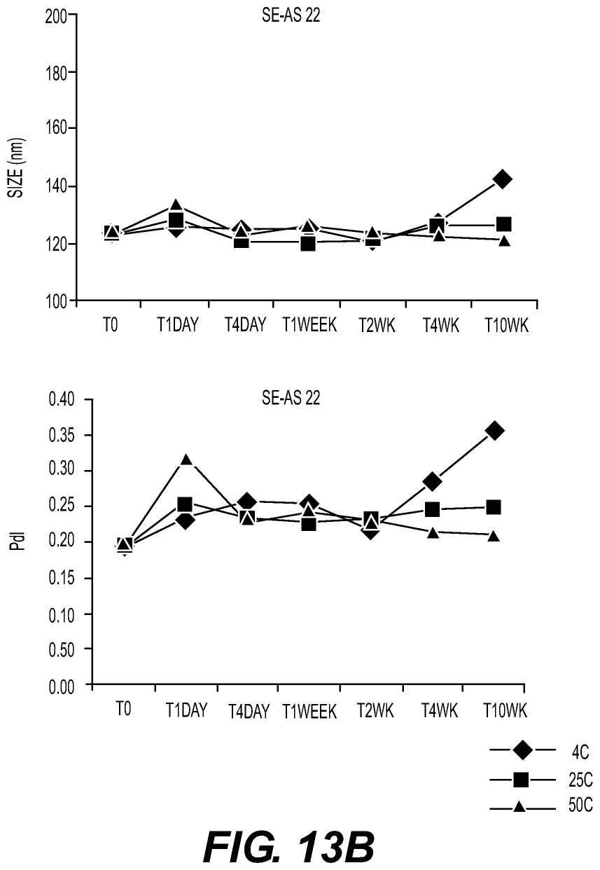

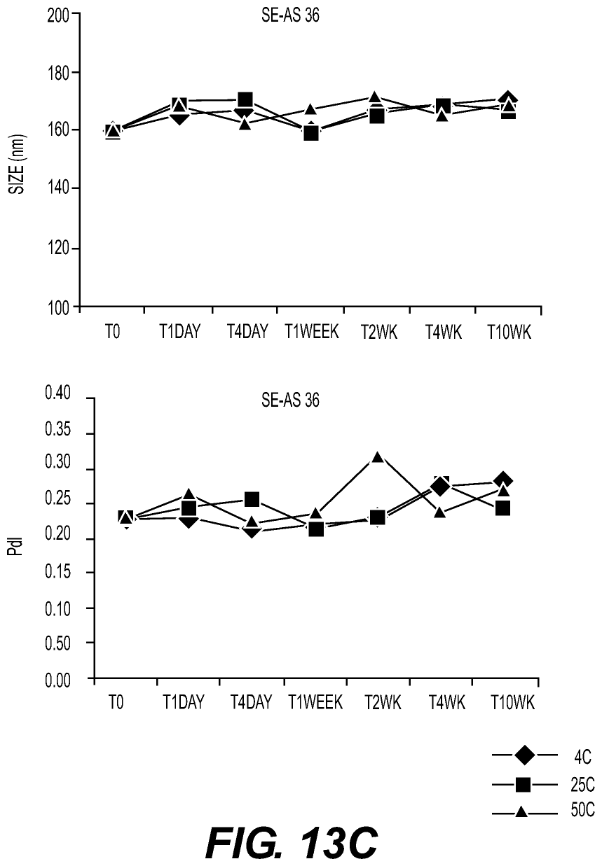

The results are presented in FIGS. 12 and 13. Changes in pH and osmolality for all three emulsions were generally limited. A decrease in pH was observed for all emulsions at 50° C. Formulation 44 droplet size and Pdl remained consistent across all temperature points, whilst the Pdl for formulations 22 and 36 fluctuated over time. The results indicate that formulation 44 is a stable self-emulsified formulation that can be sterile filtered without notably altering any physicochemical properties of the emulsion.

### Example 6—In Vivo Evaluation of Potency of Formulation 44 Compared to MF59, AS03 and SEA160 Using a CMV Antigen

Formulation 44 was tested with Cytomegalovirus (CMV) pentamer antigen (Penta) in an in vivo study.

### Method

The study design was as shown in Tables 7 and 8. Three intramuscular immunisations were performed at three-week intervals. Animals were bled at 3wp1, 3wp2 and 3wp3. Half of the animals were sacrificed at 3wp3 and the remaining at 4wp3. For both time points spleens were harvested and a CMV neutralising antibody titer assay was performed.

Concentrated emulsions were diluted with an equal volume of aqueous antigen to provide the final emulsion adjuvanted vaccine formulations. Fractional emulsion doses were prepared by initial 10-fold dilution of the concentrated emulsion, before being diluted with an equal volume of aqueous antigen to provide the final fractional adjuvanted vaccine formulations.

TABLE 7
Study design of Penta antigen at 0.05 ug dose

| Group | Treatment | No. of animals |
|---|---|---|
| A | Physiological Saline | 10 |
| | (Negative control) | |
| B | Unadjuvanted Penta | 10 |
| C | 0.05 ug Penta + formulation | 10 |
| | 44 | |
| D | 0.05 ug Penta + AS03 | 10 |
| E | 0.05 ug Penta + SEA160 | 10 |
| F | 0.05 ug Penta + 1/10^{th} | 10 |
| | formulation 44 | |
| G | 0.05 ug Penta + 1/10^{th} AS03 | 10 |
| H | 0.05 ug Penta + 1/10^{th} | 10 |
| | SEA160 | |
| I | Penta + MF59 | 10 |

TABLE 8
Timeline for study

| Day | Procedure |
|---|---|
| 1 | First immunization |
| 22 | 3pw1 Bleed |
| | Second immunization |
| 43 | 3pw2 Bleed |
| | Third immunization |
| 64 | 3wp3 Bleed |
| | Terminal bleed and spleen harvest from 5 |
| | animals per group |
| 71 | Terminal bleed and spleen harvest from |
| | remaining animals |

Formulations were administered intramuscularly (50 ul per immunisation, alternate quadriceps). 10 female 5-7 week old C57BL/6 mice were used in each test group.

### CMV Neutralizing Antibody Assay

Retinal pigment epithelial cell line (ARPE-19) was used. On day 1, 100 uL of ARPE-19 cells were plated in 96 well flat bottom plates in complete growth medium i.e. DMEM+10% FBS+1% Pen-Streptomycin. Plated were incubated in 37° C. overnight. On day 2, Tecan, a liquid handling robot was used to perform serum dilutions. Different starting dilutions were used for different time-points depending on the expected titers. A positive control from Sera care known to neutralize TB40 virus was used in every plate at a constant 1:50 dilution. In each plate, 75 uL of serum dilutions were prepared using Tecan and then 75 uL of TB40 virus was added to each well to make a total of 150 in each plate. This virus-serum mixture was incubated at 37° C., 5% CO₂ for 2 hours.

The cells plates were removed from the incubator. Media was taken out from each well and 50 uL of virus-serum cocktail was added. These plates were incubated at 37° C., 5% CO₂ for at least 20 hours. On day 3, the cells were fixed using 4% paraformaldehyde and incubated at room temperature for 20 mins following by 1 wash using 1×PBS and then permeabalized using 0.1% TritonX-100 and incubated for another 10 mins. Primary antibody (anti-mouse anti-CMV IE monoclonal antibody) was added immediately and incubated for 1 hour in 37° C., 5% CO₂ incubator. Cells were washed twice and then secondary antibody (anti-mouse AlexaFlour488 antibody) was added and incubated for another 1 hour. Post incubation cells were washed 3 times and 1×PBS was added. These plates were then read using high content imaging—CX7 (by selecting to read 10 fields per well). The selected object count is obtained from CX7 as raw data. Processing of the data gives 50% interpolated titers or EC₅₀. The final interpolated titers are then plotted and analyzed using GraphPad prism. In events where titers were low or high according to the endpoint titer analysis, the dilution was either lowered or increased. The lowest dilution was 1:50. When the serum showed titers “LOW” after 1:50 dilution, they were given a value of 25 (½ of the lowest dilution). Analysis was performed using one-way ANOVA followed by Tukey's test for multiple comparisons.

### Results

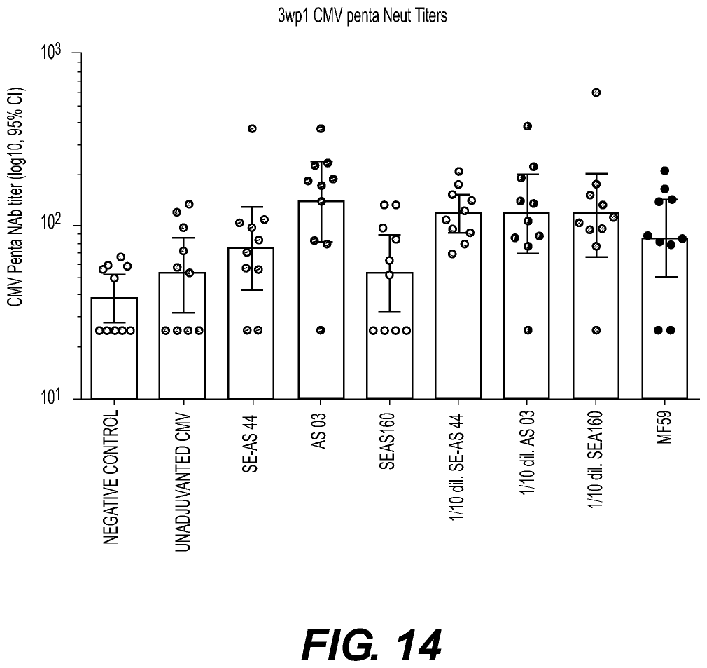

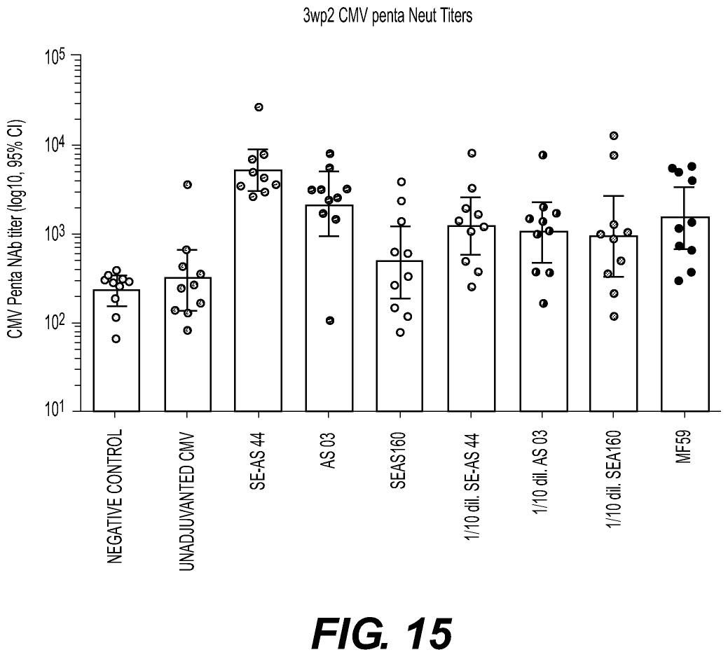

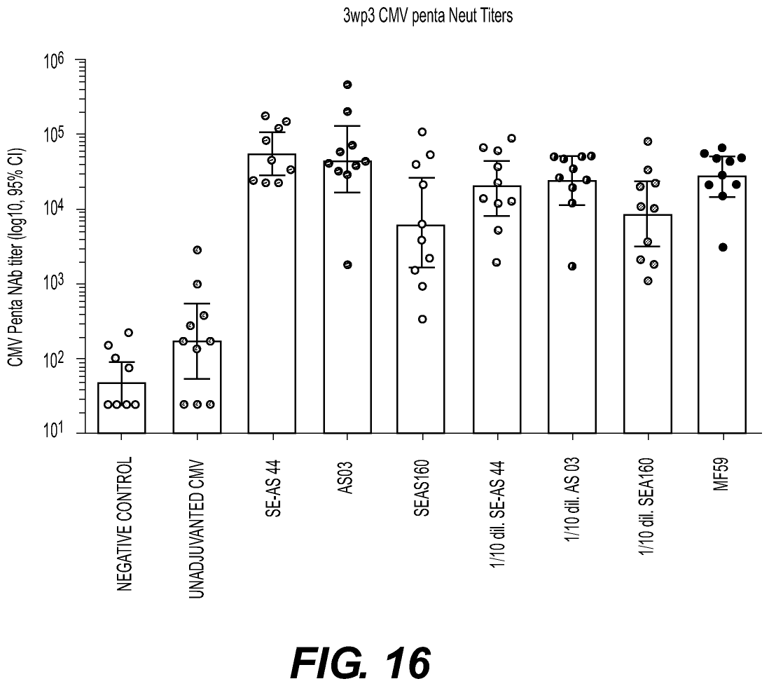

The results are presented in FIGS. 14, 15 and 16. Formulation 44 generated a significantly more potent response than the unadjuvanted group and SEA160 at 3wp2 time-point. Additionally, formulation 44 was not statistically different from AS03. Formulation 44 and AS03 generated a similar profile (FIG. 16), whilst formulation 44 gave a higher response than MF59. At this time point, formulation 44 also showed significantly better titers compared to the 1/10^{th} diluted formulation 44 group.

The results show that emulsions of the invention containing tocopherol can provide improved immunological responses as compared to formulations which do not contain tocopherol.

### Example 7—Lyophilisation Studies

Formulation 44 (SE-AS44) was lyophilised on a SP Scientific Lyostar3, which enables control over the temperature, pressure and freeze-drying cycle throughout the lyophilisation process.

### Example 7a—Initial Optimisation of Lyophilisation Composition

100 ul formulation 44, 200 ul diluent (10% w/v sucrose solution) and 100 ul antigen (OVA in DPBS, if present) or DPBS buffer alone (if no antigen present) was filled in 3 mL vials such that each contained 400 uL fill volume, with or without antigen.

Vials were equilibrated at 5° C. in the beginning of the cycle and then frozen to −5° C. to ensure standard frozen cakes in the tray. The vials were then further frozen below collapse temperature and Tg′ to −45° C. and maintained for 2 hours before pulling the vacuum and drying the formulation at −35° C. (below Tg′ and collapse temperature) until the sublimation of the ice from formulations was complete. After which, secondary drying commences which involved drying the product by removing all residual ice/water in the cake.

The lyophilisation cycle used is summarised in Table 11.

TABLE 11
Lyophilisation cycle

| | Temp | Ramp | Hold | Vacuum |
|---|---|---|---|---|
| Step | ° C. | (°/min) | (hours) | (mTorr) |
| Freezing | 5 | 1 | 0.5 | — |
| | −5 | 1 | 0.5 | — |
| | −45 | 0.1 | 2 | — |
| Primary Drying | −35 | 0.1 | — | 55 |
| Secondary | 25 | 0.1 | 6 | 55 |
| Drying | | | | |
| Hold | 5 | 1 | Until removed | — |

Lyo cake was reconstituted with 200 ul water, to obtain a 1:1 equivalent mix of antigen and adjuvant or in case of adjuvant only, a dilution to final adjuvant concentration. pH, osmolality, size and Pdl were measured after reconstitution to determine stability. Protein bis-tris gels were run to ensure antigen integrity.

TABLE 12
Lyo formulation components

| Antigen or | Concentarted | Lyo- | Total fill | Reconstituted |
|---|---|---|---|---|
| buffer | Adjuvant | protectant | volume | volume |
| 100 uL | 100 uL | 200 uL | 400 uL | 200 uL |

It was observed that the osmolality was very high for these reconstituted formulations (Table 13), due to the presence of both sucrose in the diluent as well as buffer in other components.

TABLE 13
Initial lyophilization of SE-AS 44
SE-AS 44 characterization

| | As is | |
|---|---|---|
| | (concentrated | After |
| Parameter | adjuvant) | Lyo |
| pH | 6.8 | 7 |
| Osmolality | 320-340 | 840-880 |
| (mOsm/Kg) | | |
| Size (nm) | 125-135 | 145-160 |
| Pdl | 0.15-0.25 | 0.2-0.3 |

There was a marginal size (20-25 nm) and Pdl (0.025-0.05 units) increase observed by DLS (Orr, 2014).

### Example 7b—Lyophilisation with CMY Penta Antigen

Formulation 44 was then lyophilised with CMV pentamer as a model antigen (2 ug/ml stock antigen solution) in a single vial using the LyoStar3 lyophiliser. The composition for lyophilization was optimized by formulating antigen and emulsion with 10 mM potassium phosphate buffer (in place of DPBS) to remove the salt component and obtain an appropriate the isotonicity on reconstitution. The secondary drying temperature was lowered to 25° C. to make the cycle more robust for heat-sensitive antigens.

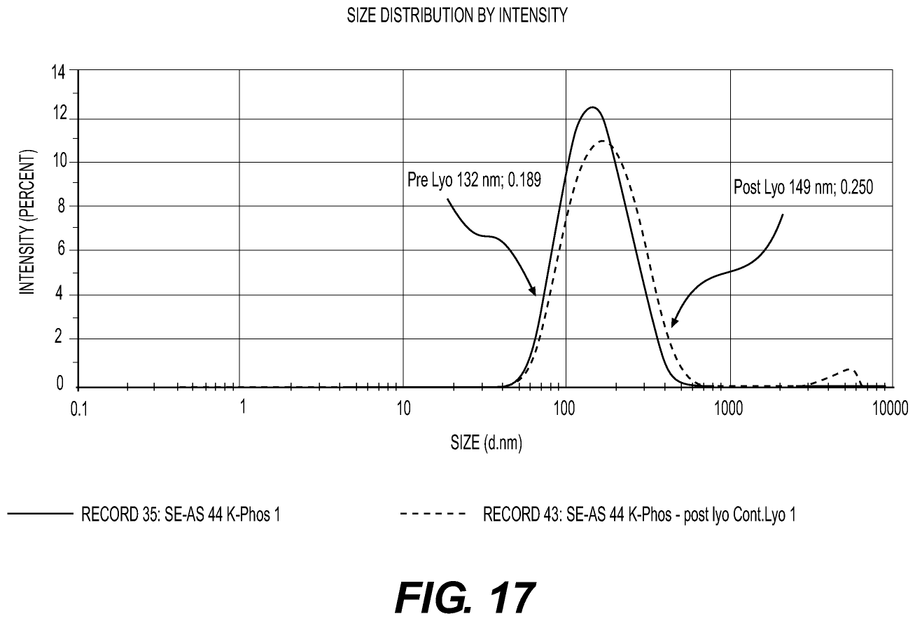

The results for lyophilisation of formulation 44 in 10 mM potassium phosphate buffer (referred to as formulation 44b) are shown in Table 14 and FIG. 17.

TABLE 14
Optimized SE-AS 44 Lyophilization
Optimized SE-AS 44 lyo

| Parameter | As is | After Lyo |
|---|---|---|
| pH | 6.8 | 7 |
| Osmolality | 320-340 | 320-340 |
| (mOsm/Kg) | | |
| Size (nm) | 125-135 | 145-160 |
| Pdl | 0.15-0.25 | 0.2-0.3 |

Osmolality remained approximately the same before and after lyophilization. The results show that there is a small increase in emulsion size and polydispersity following reconstitution post-lyophilisation.

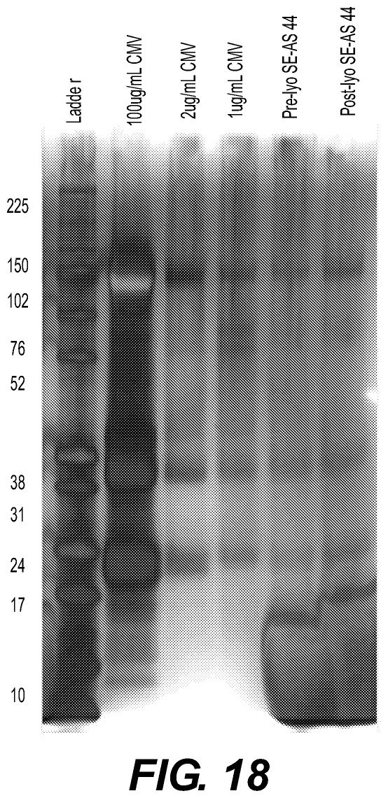

As there is no decrease in pH during lyophilisation, the protein is protected and does not undergo clipping. CMV pentamer formulated maintained its integrity post lyophilization (FIG. 18). For comparison, stock CMV pentamer solution 100 ug/mL, 2 ug/mL and 1 ug/ml are shown.

### Example 8—In Vivo Evaluation of Potency of Lyophilised Formulation 44

Lyophilised Formulation 44, prepared according to Example 7b, was tested with Cytomegalovirus (CMV) pentamer antigen (‘Penta’) in an in vivo study.

### Method

The study design was as shown in Tables 15 and 16. Three intramuscular immunisations were performed at three-week intervals. Animals were bled at 3wp1, 3wp2 and 3wp3. Half of the animals were sacrificed at 3wp3 and the remaining at 4wp3.

Concentrated emulsions were diluted with an equal volume of aqueous antigen to provide the final emulsion adjuvanted vaccine formulations.

TABLE 15
Study design of Penta antigen at 0.05 ug dose

| Group | Treatment | No. of animals |
|---|---|---|
| A | Unadjuvanted Penta | 13 |
| B | 0.05 ug Penta + formulation | 13 |
| | 44 | |
| C | 0.05 ug Penta + AS03 | 13 |
| D | 0.05 ug Penta + formulation | 13 |
| | 44 (lyophilised) | |

TABLE 16
Timeline for study

| Day | Procedure |
|---|---|
| 1 | First immunization |
| 22 | 3pw1 Bleed |
| | Second immunization |
| 43 | 3pw2 Bleed |
| | Third immunization |
| 64 | 3wp3 Bleed |
| | Terminal bleed and spleen harvest from 7 |
| | animals per group |
| 71 | Terminal bleed and spleen harvest from |
| | remaining animals |

Formulations were administered intramuscularly (50 ul per immunisation, alternate quadriceps). 13 female 6-8 week old C57BL/6 mice were used in each test group.

CMV neutralizing antibody assay was performed essentially as described in Example 6.

### CMV IgG Assay

Antibody titers were determined in serum obtained from each animal at 3wp2 and 3wp3. To determine the CMV pentamer specific binding IgG antibody titers, sandwich ELISA was used. 96 well Nunc-immuno Maxisorp F96 plates were used to coat 100 ul of 1 ug/ml CMV pentamer antigen per well overnight at 4° C. Antigen coated plates were washed with 1× phosphate buffered saline (PBS) & 0.05% w/v Tween20 and blocked with 1% w/v bovine serum albumin (BSA) solution in PBS. Serum from immunized animals was added in the first row of the plate such that well A1 received positive control and well A12 received sample buffer as negative control. The serum was prediluted before adding 10 ul to row 1. Serial dilution was the performed down the plate from row A to H. Serum incubation was allowed for one hour before washing the plates and adding horse radish peroxidase (HRP) conjugated goat anti-mouse IgG from Jackson lmmunoresearch (West Grove, Pa.) for another one-hour incubation at room temperature. Substrate was added quickly after washing plates again, for 15 mins and then immediately stop solution was added. Plates were read using En Vision 2105 Multimode plate reader from Perkin Elmer (Waltham, Mass.). Titers were calculated at 50% interpolated optical density (OD) value obtained from the plate reader.

### ICS

T-cell responses were analysed 4wp3 by intracellular cytokine staining of in vitro antigen-stimulated splenocytes. Spleens from individual animals were processed to single-cell suspensions, followed by treatment with RBC lysis buffer (Ebioscience, Thermo Fisher Waltham, Mass.). CMV pentamer peptides gH, gL, UL128, UL130 and UL131 from GeneScript (were used for stimulation of splenocytes. These splenocytes were stimulated at one million cells per well density with anti-CD3 from BD Biosciences (San Jose Calif.) used as positive control, media was used as negative control, and peptide pool was prepared for antigen stimulation condition. Anti-CD28 antibody from BD Biosciences was added to each well as a co-stimulant and brefeldin A (BFA) from BD Biosciences was added two hours after stimulation at 1 ug/ml concentration for blocking cytokine secretion. The cells were stimulated overnight and stained with live/dead reagent (Near IR, EX 633/EM 750). Before the cells were fixed and permeabilized using Cytofix/Cytoperm reagent, Fc block was added to avoid extracellular non-specific binding, followed by memory marker staining using CD62L conjugated with BV510 and CD127 conjugated with BV421 from BD Biosciences. Fc block was again added to avoid intracellular non-specific binding before single-step staining with CD3 conjugated with BV711, IL-17F conjugated with AF647 from BioLegend (San Diego, Calif.), CD4 conjugated with BUV395, CD8 conjugated with BB700, CD44 conjugated with PEFC594, Interleukin 2 (IL-2) conjugated with APCR700, Interferon γ (IFN-γ) conjugated with BV786, tissue necrotic factor α (TNF-α) conjugated with BV650, IL-17A conjugated with BV421 from BD Biosciences, and IL-13 and IL-4 conjugated with AF488 obtained from Thermo fisher Scientific (Waltham, Mass.). Since most of the anti-mouse antibodies used are rat or hamster derived; anti-rat anti-hamster Ig, K/Negative control compensation particles from BD Biosciences stained with all the above fluorochrome conjugated antibodies including an unstained control for preparing compensation controls. The samples were acquired on a BD FortessaX20 SORP flow cytometer from BD Biosciences (San Jose, Calif.) followed by analysis with FlowJo software (Ashland, Oreg.).

### Statistics

GraphPad Prism software (San Diego, Calif.) was used to analyse and plot data from the in vivo immune responses. For humoral responses analysis was performed using one-way ANOVA followed by Tukey's test for multiple comparisons. Non-inferiority compared to AS03 for HAI titers was tested by running Dunnett's test post one-way ANOVA. For ICS, a nonparametric Kruskal-Wallis test was run followed by Dunn's multiple comparisons test for comparison within different dosing groups.

### Results

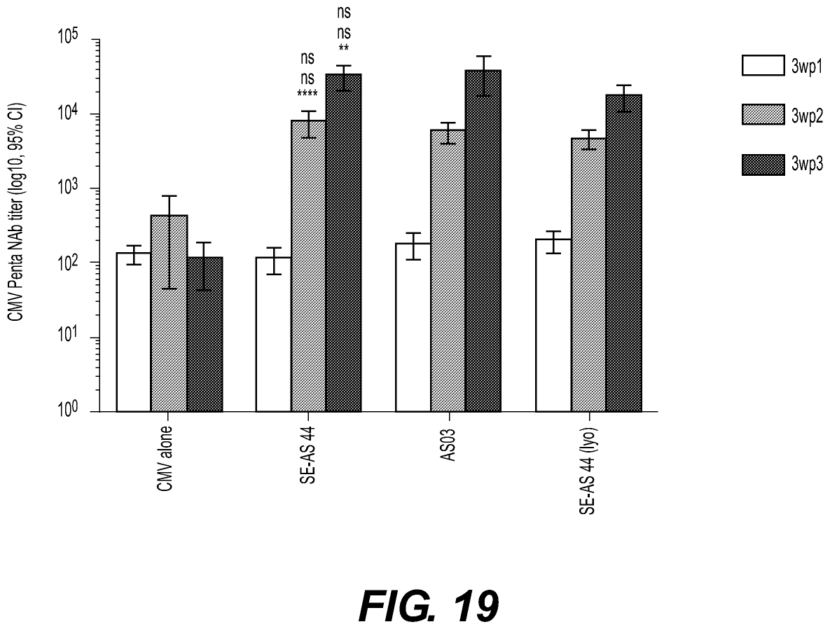

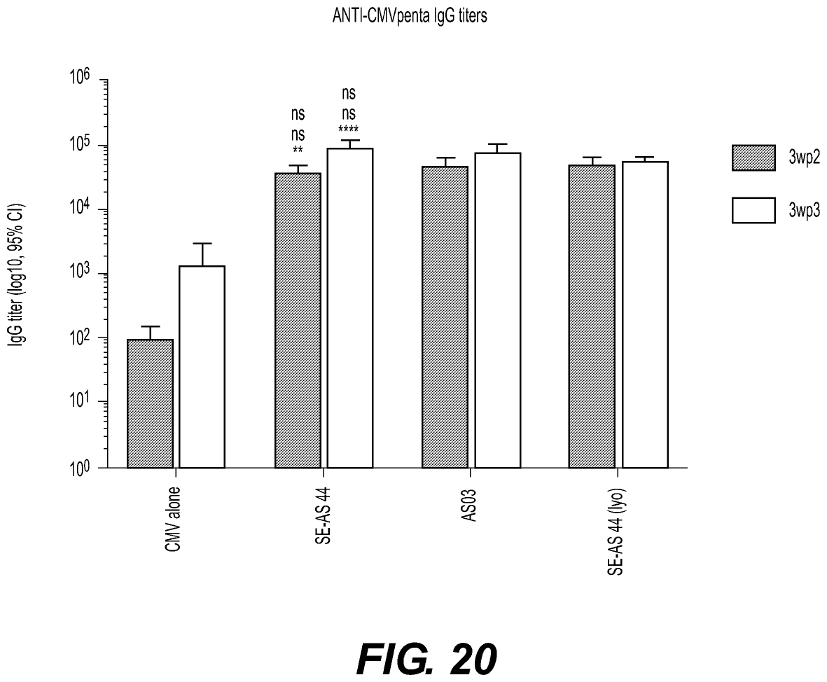

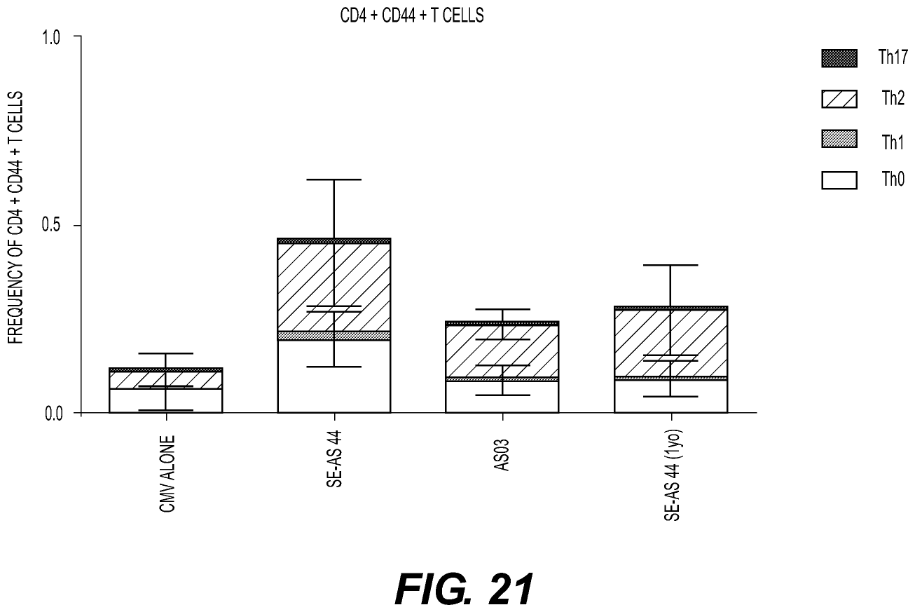

The results are presented in FIGS. 19, 20 and 21.

No significant difference was observed between neutralising antibody titers or IgG antibody titers for the liquid or lyophilised and reconstituted preparations of Formulation 44. Additionally, both the formulations were comparable to AS03.

ICS showed a dominant Th0/Th2 response. Lyophilized vaccine showed no significant difference with liquid vaccine or with AS03.

### REFERENCES

Banzhoff Immunology Letters 2000 71:91-96
Chandramouli et al Sci. Immunol. 2017 2 eaan1457
Fox Molecules 2009 14:3286-3312
Franchini et al. Poult Sci. 1991 70(8):1709-1715
Franchini et al. Poult Sci. 1995 74(4):666-671
Garcon et al. Expert Rev Vaccines 2012 11:349-66.
Jackson Journal of the American Medical Association 2015 314(3):237-46. doi: 10.1001/jama.2015.7916
Julianto et al. International Journal of Pharmaceutics 2000 200:53-57
Lidgate et al Pharmaceutical Research 1992 9(7):860-863.
Lodaya et al. Journal of Controlled Release 2019 316:12-21
Morel et al. Vaccine 2011 29:2461-2473
O'Hagan Expert Rev Vaccines 2007 6(5):699-710
O'Hagan et al Expert Rev Vaccines 2013 12(1):13-30
Orr et al Journal of Controlled Release 2014 177 (Supplement C):20-26
Podda Vaccine 2001 19: 2673-2680
Podda & Del Giudice Expert Rev Vaccines 2003 2:197-203
Shah et al Journal of Pharmaceutical Sciences 2015 104:1352-1361
Shah et al Nanomedicine (Lond.) 2014 9(17), 2671-2681
Light Scattering from Polymer Solutions and Nanoparticle Dispersions (W. Schartl), 2007. ISBN: 978-3-540-71950-2
Handbook of Pharmaceutical Excipients (eds. Rowe, Sheskey, & Quinn; 6th edition, 2009).
Vaccine Design: The Subunit and Adjuvant Approach (eds. Powell & Newman) Plenum Press 1995. ISBN 0-306-44867-X
Vaccine Adjuvants: Preparation Methods and Research Protocols (Volume 42 of Methods in Molecular Medicine series). Ed. O'Hagan. ISBN: 1-59259-083-7
New Generation Vaccines (eds. Levine et al.). 3rd edition, 2004. ISBN 0-8247-4071-8

1. A composition comprising squalene, a tocopherol and a biocompatible metabolisable surfactant, wherein the squalene is 40% v/v or more of the composition, the tocopherol is 25% v/v or less of the composition, the surfactant is 60% v/v or less of the composition, which when mixed with an excess volume of substantially surfactant-free aqueous material, forms an adjuvant having an average oil particle diameter of 200 nm or less.

2-4. (canceled)

5. A composition comprising squalene, a tocopherol and a biocompatible metabolisable surfactant, wherein the squalene is 50 to 70% v/v of the composition, the tocopherol is 10 to 20% v/v of the composition and the surfactant is 10 to 40% v/v of the composition.

6. The composition according to claim 5, wherein the squalene is 55 to 65% v/v of the composition, the tocopherol is 10 to 20% v/v of the composition and the surfactant is 20 to 30% v/v of the composition.

7-8. (canceled)

9. A method for preparing an oil-in-water emulsion adjuvant comprising squalene, a tocopherol, a biocompatible metabolisable surfactant and an aqueous component, said method comprising mixing a composition according to claim 1 with an excess volume of an aqueous component.

10. The method of claim 9, wherein the aqueous component comprises an antigen or antigenic component.

11. The method of claim 10, further comprising a step of drying the oil-in-water emulsion, such as by lyophilisation.

12. (canceled)

13. A dried composition obtainable by the method of claim 11.

14. An oil-in-water emulsion adjuvant composition comprising squalene, a tocopherol, a biocompatible metabolisable surfactant and an excess volume of an aqueous component, wherein the squalene is 40% v/v or more of the total amount of squalene, tocopherol and surfactant, the tocopherol is 25% v/v or less of the of the total amount of squalene, tocopherol and surfactant, the surfactant is 60% v/v or less of the total amount of squalene, tocopherol and surfactant and wherein the adjuvant has an average oil particle diameter of 200 nm or less.

15-17. (canceled)

18. An oil-in-water emulsion adjuvant composition comprising squalene, a tocopherol, a biocompatible metabolisable surfactant and an excess volume of an aqueous component, wherein the squalene is 50 to 70% v/v of the total amount of squalene, tocopherol and surfactant, the tocopherol is 10 to 20% v/v of the total amount of squalene, tocopherol and surfactant, the surfactant is 10 to 40% v/v of the total amount of squalene, tocopherol and surfactant.

19. The composition according to claim 18, wherein the squalene is 55 to 65% v/v of the composition, the tocopherol is 10 to 20% v/v of the composition and the surfactant is 20 to 30% v/v of the composition.

20. The composition according to claim 18, having an average oil particle diameter of 200 nm or less.

21. The composition according to claim 14, comprising at least 80% v/v water, such as at least 85% or at least 90%.

22. (canceled)

23. A vaccine composition obtainable by the method of claim 10.

24. The composition according to claim 14 further comprising an antigen or antigenic component.

25. The composition according to claim 18 further comprising an antigen or antigenic component.

26. The composition according to claim 1, wherein the polydispersion index of the adjuvant is 0.3 or less.

27. The composition according to claim 1, wherein the tocopherol is alpha-tocopherol.

28. (canceled)

29. The composition or method according to claim 14, wherein the surfactant comprises polysorbate 80.

30. A vaccine kit comprising
an antigen or antigenic component; an aqueous component and a composition according to claim 1.

31. The vaccine kit of claim 30, wherein either or both of the aqueous component and the composition comprise the antigen or antigenic component
